SEQUENCE LISTING (39 sequences)

SEQ ID NO: 1 - DNA, 349 bp, Ixodes scapularis
atggaaatatctgtgaaaccaaggcccacaaaaaggaaaagaaaggccatcatcatcatggcaagaatgagaacagcattccccaccagaagtgggaacagcttctcaaggacttgaacagttaatgatgtgttgtgcaattcgaatgtggctgcaacctcgctagagaacatagtcgaccagctgtagtgctctagtattaaccaagaagcagtattctgccgtcatatgtacaggcagatttgttacggcattttcagcttttttttatacaaaatgtagttcttgtttaaaaaaaacctaaaataaaacaaagccacaaaaaaaaaaaaaaaaaaaaaaaaaaaaa

SEQ ID NO: 2 - protein, 38 aa, Ixodes scapularis
MEISVKPRPTKRKRKAIIIMARMRTAFPTRSGNSFSRT

SEQ ID NO: 3 - DNA, 2693 bp, Ixodes scapularis
  misc_feature (685)..(685): n is a, c, g, or t
ggtttgtgctgcttggaaaatccaccaggagcctgcaaccgcaaaaaagttcatcatccggagctaagcctatcgcaggatggcttgcgcaacattaaagcgaacacacgattgggatccgctgcatagtccaaacggaagatcgcccaaacgacggagatgtatgcctttgtcggtcacacaagcagcgactcccccaacaagggcacaccaaatcaacccttcacccttcggtgaagtgccaccgaaattaacttcagaggagatagcggccaacattcgggaggaaatgcgacgtctgcagcggcgcaagcagctctgcttctcgtctcccctggagtcgggctccccgtcggcgactccccctgcggccgattgcggaccagcctcccccacgggcctgtcccccgggggcctgctgtcgcccgtgcgcagggaccaacccctcttcaccttccgccaggtggggctcatctgcgagcgcatgatgaaggagcgcgagagccagatacgcgacgagtacgaccacgttctgtctgccaagctggcagagcagtacgacacatttgtcaagtttacgtacgaccaaattcagaagcggtttgagggtgccactccaagctatttgtcataacatgatgggcatctgcaaacaagcaaggaactttgagggtttgtgctaganggaagaaacccatggtggggaaggacacaagaccaacacttagactcggcaagcaagccagatcctgtggggtgcggggacggggggaatgagtccagtggtgtcttcggagtttttttttttccttctccctttccctcgtcttctttttggcacaactctttacggaactggtgtgcatccattccccgaaagtgcaagagaaggactcgcggcggatcatctacggaggaagaagtgtgtatgcctttgtgctttgggtctccttttttttttttttaaccgtcttgccatctcgccatagaagacctgtgatctagcaaacaaaggtgtgcgaatgttatgcaaaggttggaagtcagtttgaaagtggagcgagagaaaattttgtatgctgagtatggttagtcaccgtttacttttcaggagggggatgactgaggaacggagccgccccaactctcgtttgtcttttatttttaggatactttctctgtggcgagaatttgtgtgtgcatgcaagttagcgagggtacgaggaaaagaagggttataaaatattctgggtgagagctgtagttcaactggggggtgggattgtaaagacctgcgggtaccgagaggaccgcatgctctggctatattacttgcattgagggggaggaggaatgctggacctcgagcagagccagcaagtattttggaaaggaataaaaacaaaaattggcttagtgtacagatgtataatatatatgcactacagggtgtgtgtgcctcttgtatcttcccgtgcgttgtgtccctcttgcggcttgcccatctgacaaccgcctgtgtacataggccaacgcaagtcttcagcatggcaccctcttctttttccttttttttttctcataagtaattttgaaggagagaatattttgatttctaagactcccaaaacatcaagtgctctggtggtcggaattctacaagtgcgaaagttcctttcttttttttgtttcgagataggaatggcttcaggttgtgctgcctatgctttggccacactttggaacacctgcaacagcgaattaactggtgtaggcctgtgacacttgcacagccgtgttttttttttttttttttgtagttttgcagtaataaaaacttgttatggaaagagtgcattatgctatggcattgtctgctgctatgcttattggaatgcatgcctgatgtgtgttgtgcttgaggatagtgaagtggtattgcagggttggaaaggagcttanaatgccttctggcttttgcataagcgtggctttgggtgtcgtctgagcttgtcaatcacagtgcaacatgcactttgtccaattggtttattggggactgcttttgggtgcagagtttgactaatttttagtaatgcttcaaatgcaacgcttctgtgttgatcgcagttcatcaactcgtcgatcattatgcatgtgaaaaactgctcacgtaaactgtatgttgatatcacagttgcactgaggaagcctggcttaagatggtgtgtgcaagtgcttggcacactgcgtattttccagcataaagctggtagtgtacaggtgctgctgttagtagcaaactttctgccattgctgccacaattcatgcatgaatgagtgttggggagtatgttagtttatcttttcaaaactgatttgaagtaccagtgtcctatatttttgccattgcattaatatggatcctgcattgtttacggaaaaaagtgataacattaattatgaaagatattaagcgagtttattgacttttccaggagaatttagaccaggaggcactacatagcctgtggtctgctttgttatgttgacttggtttttgtggaaattagttctaaaagtttacaatctttttggcatgacttgtttgcattgccattgtaatttggccattattagaataaaggcactctctcagtacctaaaaaaaaaaaaaaaaaaaaaaaaaaaaa

SEQ ID NO: 4 - protein, 184 aa, Ixodes scapularis
MACATLKRTHDWDPLHSPNGRSPKRRRCMPLSVTQAATPPTRAHQINPSPFGEVPPKLTSEEIAANIREEMRRLQRRKQLCFSSPLESGSPSATPPAADCGPASPTGLSPGGLLSPVRRDQPLFTFRQVGLICERMMKERESQIRDEYDHVLSAKLAEQYDTFVKFTYDQIQKRFEGATPSYLS

SEQ ID NO: 5 - DNA, 1821 bp, Ixodes scapularis
  misc_feature (1487)..(1487): n is a, c, g, or t
atggcgtcgtgtggagcatcagcgacgggtcctctcgtcctaagagtaatttccaacactgttaaaatagttaacagcgccggaaagataatcaaggacatcatgaacagtggaaacctcggaatcgtcgaaaaggaaggcatcaatgacctgcaaacggaggcagacagatctgttcagcgctgcattgtgacttcgctctcgagacagttcccaaaactgacaataattggtgaagagactctggaggagaaaaagatcagcgacgactggatcatcaccgagcatgacaaggatgtcctggccacttctctgccggacaacctgaagaacatcaaagaggaagatttggtagtctgggttgatcctctggatggaaccaaggagtacacacagggtttcctggaccacgtgacgatcctggtggggattgcggttgacggtaaggcagtgggtggagtgatccaccagccgtactacaactaccaggtggagaaggacgtctacaagcagggacgtaccatgtggggcattgtcggcgtcggtgcctttgggatctcgcgcattgcgcctccggagaacaagaggatcatcactacgacgcgctcccattccagccccaccatcaacagctgcattgaagccatgaatccggacgaggtgctgcgagttggaggtgccgggcacaaggtgctgctgttgattgagggcaaggctcacgcttacgtgtttcccagcaaagggtgcaagaaatgggacacttgtgcccccgaagcgattcttcatgccactggcggccttcttacagatgttcacgggaacagattggagtaccacaaggacgtggaacacgtcaatgccggcggcgttcttgccacctgcctgaaggaacaacacgaatggttcaagaaccacattcccgaagatgtccgcaagacgcttcctctatgagcaacctgccgttgtccgttgcgatcacactcaagtcgcgtttttcctttaactttgtggtgatgcggttcaaagtcttatactattagtgttttggtggtccaaatattattactaaaaaaacccggagacatgggacacaaaaaaatggaggggcgggacaataaggtctcgaacacagctcgtacagaattttttaaaataatgttgatttcaggtttatttgtggaaactctgaaattaaccgttatgtcattatttgggttgtgccgtttgaaattttatgaaatacgtaatagctgcacgcattttgcaggccactcagctccttgaatgctcgatgcttgatgcttctgccaacattatttgtatctcaagtttttctaccacaagaaacagtaccctaacattttgaaatagtattactagcttggattttatctggtatgcatatataagatctatggatgttcctaaggagggcatgaatttgaaacataccctgtccttaccaaccttcaaacatttttttttgngcctgcttaaaagcacttacattgcttgatcgttgaattaattttttagctgatgttaaggacacttataataattaaggaaatgagatcgatcttgagcttgtttgngcctctgtaanaattgatgctctttcanacctaatgcttaatgcaacaataattatcaagtaatccttactcaggtgtcagatatgcaagcagatgccaatgcttctgttcattgagtggcaaaaggcattgctctttgtcacattgcatgcatttatgacagcccnccttaataaactataatgcagctaatntgaaaaaaaaaaaaaaaaaaaaaaaaaaaaaa

SEQ ID NO: 6 - protein, 316 aa, Ixodes scapularis
MASCGASATGPLVLRVISNTVKIVNSAGKIIKDIMNSGNLGIVEKEGINDLQTEADRSVQRCIVTSLSRQFPKLTIIGEETLEEKKISDDWIITEHDKDVLATSLPDNLKNIKEEDLVVWVDPLDGTKEYTQGFLDHVTILVGIAVDGKAVGGVIHQPYYNYQVEKDVYKQGRTMWGIVGVGAFGISRIAPPENKRIITTTRSHSSPTINSCIEAMNPDEVLRVGGAGHKVLLLIEGKAHAYVFPSKGCKKWDTCAPEAILHATGGLLTDVHGNRLEYHKDVEHVNAGGVLATCLKEQHEWFKNHIPEDVRKTLPL

SEQ ID NO: 7 - DNA, 697 bp, Ixodes scapularis
  misc_feature (573)..(573): n is a, c, g, or t
gacctcaaggaaggcatctgcccgcaggccttctggctcaacaaggagcagtgttgctgggcctccaacgataccttctttaagggggacgactgcaagcagtggtatcggtggcccgagatgttcgacagcggcatggacaaggacggggcaggcttttacctgctctcctacctgctgtacgtcatgtggagtgtgctcttcgccaccctggccgtcatgctcgttcgcaccttcgcgccctatgcctgtggatctggaatcccggagatcaagacgattctgagcggcttcatcatccgcggctacctgggcaagtggacgctgaccatcaaatcagtgtgtctggtgctggccgtcggggcgggcctcagcctgggcaaagaggggcccctggtgcacgtggcctgctgcatcgggaacatcttctcctacctcttccccaagtacggcaagaatgaggccaagaagagggagatcctgtcggctgccgccgccgcgggagtttctgtggcctttggggctcccatcggcggtgttctcttcagcctcgaagaggtgagctactacttncccttgaagacgctgtggcgttccttcttctgcgccctggtggcagcctcggtgctgcgctccatcaacccctttggcaacgaccacctggtgatgttctacgtcgagtacgactttccctggc

SEQ ID NO: 8 - protein, 232 aa, Ixodes scapularis
  misc_feature (191)..(191): Xaa can be any naturally occurring amino acid
DLKEGICPQAFWLNKEQCCWASNDTFFKGDDCKQWYRWPEMFDSGMDKDGAGFYLLSYLLYVMWSVLFATLAVMLVRTFAPYACGSGIPEIKTILSGFIIRGYLGKWTLTIKSVCLVLAVGAGLSLGKEGPLVHVACCIGNIFSYLFPKYGKNEAKKREILSAAAAAGVSVAFGAPIGGVLFSLEEVSYYXPLKTLWRSFFCALVAASVLRSINPFGNDHLVMFYVEYDFPW

SEQ ID NO: 9 - DNA, 1221 bp, Ixodes scapularis
  misc_feature (713)..(713): n is a, c, g, or t
gtttcttgttacggtagtggagtgctgagtttactcgataatatctgagaaatagtggcaatatcaatttttctgtaaattagaaatgtaaccaatggcgtggctatcttctagtcgacactaacgtctcggatctgctgttcaaagtcccgggcgatcaagccgtatttgttgtccagctgccaagtgcgtcgaatatgatgccgaaaaagaaagaatcagtcgcgagctctaaagaagacgcgccgatcgacgtgatcggcctgccctcccacaaacgacacaagaagcacaagcacaaaaagcacaagcgcaagcgaggcacggaccaagacgaagaccaatcgcccgccgcgagcccgcagagcggtggcgagggtagcagcagcaagcccgcgctcaagctcaagatcaagatcggcggacagacggtcgagaagaacgtgaccaagctgaaacagcagcggccgccgccgccggaccctagcgaagccgatctcgccgaactcctgatgaaacccaactcgggcgatacgagcgcagacagcgatgacgaagaggaagcctggctcgaagccctcgagtccggcaggctcgaagaggtcgacgacgagctccgcaaaatgaaggacccgaccctgatgacggccaggcagcgggccctgctcgagagcaagtcgcagaaggacgaggtcccggcgacggggatggcnggcgtccgcggagcccgtcaaagagatgtccgaggagatgattcagcggcggatgctgcgggccaaaaagcggaagcagcaggccgaagagaagaaagagaaggagaagaagcagacgatcgagcgtctgctcaagaagtccgactcgaggctgagggccagcaagaagttggccaagaagagcgatactcccaaggtgtcgctggtcaacacgcaggcaggcacgctgctctcgtttcccgtcggcgttgcgttcccgctgtcggcagccgtggcccaggggtaccccgagaagacgacgtgcggcattaagggttgtcgtaacccgaagaagtactcgtgctccaagacaggcgtgcccctgtgcagcctcgagtgctacaagacgaacatgctgcagatgtgcgtctgagcgggcagctaggcttccgggctacagctgctccttgtgtatatgtatataaagtcgagaatgctgaaaaaaaaaaaaaaaaaaaaaaaaaaaaaa

SEQ ID NO: 10 - protein, 275 aa, Ixodes scapularis
MMPKKKESVASSKEDAPIDVIGLPSHKRHKKHKHKKHKRKRGTDQDEDQSPAASPQSGGEGSSSKPALKLKIKIGGQTVEKNVTKLKQQRPPPPDPSEADLAELLMKPNSGDTSADSDDEEEAWLEALESGRLEEVDDELRKMKDPTLMTARQRALLESKSQKDEVPATGMAGVRGARQRDVRGDDSAADAAGQKAEAAGRREEREGEEADDRASAQEVRLEAEGQQEVGQEERYSQGVAGQHAGRHAALVSRRRCVPAVGSRGPGVPREDDVRH

SEQ ID NO: 11 - DNA, 1942 bp, Ixodes scapularis
cgatgcaggcgatgacgggctttgcggtgcagttcaacaaaaacagtttcgggctgactccagctcagccgctgcagttgcagattcccctgcagcccaacttcccagctgatgcgagcttgcagctgggaaccaacggtcccgtgcagaagatggaccccctcaccaaccttcaggtggccatcaagaacaatgtggacgtgttctacttcagctgcctggtgcccatgcacgtgctgagcacggaggacggcctgatggacaagcgggtgttcctggccacctggaaagacatccccgcccaaaacgaggtccagtacaccctcgacaacgtcaacctcactgcagaccaagtttcccagaagctgcagaacaacaacattttcacgatagccaagaggaacgtggacggccaggacatgctgtaccagtccctgaagctcaccaacggcatttgggtgttggcggagctcaagatacagcccggcaatccaaggatcacgttgtctttgaagacaagagcacctgaagtggcagcaggtgtacaacaaacttacgaactcattctacacagctgaggctgctgtgaatgaaactcttctcccacccccttcttttgatggcagtcaatgtctcgtttcattttcttgttttcttttgcggcgtgctacggaacaaggtcctacattcccaagttatatggtgttgtcgcgtagggggcagagtgccgctgagcccgcgacagccttgtttctgaggagagccgaacgcaccacttcgaaaaagaaaaagtgaaaacggaaaaatgaaaaattttccagttgcttcaaattaacattcctcgtagtcagtctgtggccgttgagtttggtgtaaagaagaaaaaggtgtctcttttagtgaaaatggttgctttttattggtatcccctatcacaccgagcacgaacataagaaatcctgacaaggattctcctttagttgtattatggtggctggagcacacgaggcacctgttgccaattcgacccagcaaatgcccaattctcaagatttgagttcattgaggttgttttgctcctccccccccaccccccaactttgtcgttggattgtctaacagtgtaaatgggcgacgactcgttattctttttttcttcattctttctttttgttgtcacgcgccccgggggacgcgacacaacttatgtgcataattgattttcacaggctgcgacgcagtctgtaaaagaaggggaagtgaaactctgctccgccgctgctagtgtcatcacgggacgaccatcgcgttttctctgactatttaaacaaaactgcatagcttagggggcagtctgtgcaaagtggaacaaccaaactgagccctgccctttcggtgtgtgtacaagcatctctgtgtaacatgaactactttacatgaactacattgcatgaacgggagaagtttagttgtttttttgttttttttttcaggtgactatgtcaacagattagaaccattttttggaacggctggaaagataaccgctcattttgtttctactaaaagactacgaaaagtgttgactttttgcatcggtttggcaacgtttgtttggcatgcatgtagttgagcgtaatggtatcacccctcgtaaacaataacagtgcaatggagcagtactgtagtgtccattaaagagcgagagtttggttaaaggttgttaattgaggtccgtgttatcctttgagtaggagagcggcactttttgcaaatagcgctgctgggggcgtcatatctgccctccaaaacatgcacattttaagtgtgaattgttgcggcggcttgtacaagtatgtgtgttatgtgtagaaaaagaactcttaattaaaatatttgtggccaaaacgtcaaaaaaaaaaaaaaaaaaaaaaaaaaaaaa

SEQ ID NO: 12 - protein, 191 aa, Ixodes scapularis
MQAMTGFAVQFNKNSFGLTPAQPLQLQIPLQPNFPADASLQLGTNGPVQKMDPLTNLQVAIKNNVDVFYFSCLVPMHVLSTEDGLMDKRVFLATWKDIPAQNEVQYTLDNVNLTADQVSQKLQNNNIFTIAKRNVDGQDMLYQSLKLTNGIWVLAELKIQPGNPRITLSLKTRAPEVAAGVQQTYELILHS

SEQ ID NO: 13 - DNA, 1428 bp, Ixodes scapularis
  misc_feature (701)..(701): n is a, c, g, or t
cgcgccgtgcagaagctgcgtcgggaggttgagaaggcaaagaggaccctgtccactgctcaccaggccaggatcgagattgaatcgttcttcgagggagaggacttcagtgagaccctgactcgtgctaagtttgaggagctgaacatggaccttttccgttccaccatgaagcctgttcagaaggtactcgaggatggtgacctcaagaagactgatgtggacgagattgtgcttgtcggaggttccaccaggatccccaaggttcaacagctggtcaaggagttcttcaatggcaaggaacccacccgtggcatcaaccccgacgaagcagtcgcctacggtgccgccgtgcaggctggagtcctcggcggagaggaagacactggggacctcgtgctgttggacgtgaaccctctgaccctcggcatcgagacagtgggaggcgtcatgacgaaactgatcccccgtaacacagtcatccccacgaagaagtctcagatcttctccacggcctcggacgagcagagcactgtcaccatccaggtctttgagggggagcgtcccctgacaaaggacaaccaccagctgggcaagttcgacctgactggcatcccacctgctcctcgaggtgtgccccaaatcgaggtgaccttcgagattgacgtcaacggtatcctgcgggtcagtgcagaggacangggtacaggcaacaagcagaagatcaccatcaacaatgaccagaacaggctgacgcctgaggacatcgagaggatggtaaaggacgccgaaaagtttgccgacgaggacaagaaggtcaaggagaaggtggaggcccgcaacgaactggagtcttatgcctactccctcaagaaccagattggagacaaggagaagatgggaggcaagctctccgacgaggacaagaagactattgagcaagctgtggacgagaaaatcaaatggctggagcagcacagtgacgctgatgcggaagaactcaaggaacagaagaaacagctggctgatactgtgcagccgattgtagccaagctgtaccctgcaggaggcaccccaccgccgacggacaaagatgactctacaaaggacgagttgtaaaaacaaggccagatctcttgggtacagcgaaaggcatggggcagcagcattatcacaagtcatctgttacgatcatgagctcatcatttcaccacctctacagtgctgctgctgcctgccttttggctggttgagtgttcttggacctatttaccatgatcattctctgtacaaaaacaattctttctgtgtttttttttttttcgttgtagtaacttaagttatacagatgtcttctactgggtgggctttctccatgagtgggagggggctgggtgtcaaataaaagtgtttctattaaaaaaaaaa

SEQ ID NO: 14 - protein, 372 aa, Ixodes scapularis
  misc_feature (234)..(234): Xaa can be any naturally occurring amino acid
RAVQKLRREVEKAKRTLSTAHQARIEIESFFEGEDFSETLTRAKFEELNMDLFRSTMKPVQKVLEDGDLKKTDVDEIVLVGGSTRIPKVQQLVKEFFNGKEPTRGINPDEAVAYGAAVQAGVLGGEEDTGDLVLLDVNPLTLGIETVGGVMTKLIPRNTVIPTKKSQIFSTASDEQSTVTIQVFEGERPLTKDNHQLGKFDLTGIPPAPRGVPQIEVTFEIDVNGILRVSAEDXGTGNKQKITINNDQNRLTPEDIERMVKDAEKFADEDKKVKEKVEARNELESYAYSLKNQIGDKEKMGGKLSDEDKKTIEQAVDEKIKWLEQHSDADAEELKEQKKQLADTVQPIVAKLYPAGGTPPPTDKDDSTKDEL

SEQ ID NO: 15 - DNA, 1847 bp, Ixodes scapularis
  misc_feature (1814)..(1814): n is a, c, g, or t
cgacgtgtttgtgagtgcagcggtgaactggacggtgtcgtggccacgcgatggcagcggcggtgatgaactgcctacggactgcgcttttaggcgctctcgtcgtccaactctacgccacgcagataggtcaccggaaattcgagtacaagtacagtttcaagggaccctacctggcgcagaaggatggatcggtgcctttctgggagtacggcggcaattgcatcgccagtgaggagatggttcggatcacgccctccctgaagagcaagaaaggatccatctggtccaagctgccgacatcgttcccttggtgggaggtggagctggtgttccgcaccacgggtacgggcaggataggagctgacggcctggccttctggtacacagacaagaagcaggcggagggtcctgtctttggaagcagcgacaagtggactggcctggccatcttcttcgattccttcgacaatgataacaagcacaacaacccatacatcatgggcatggtgaacgatggaacaaaagcctacgatcatgagagtgacggtgccaaccaacagctagcgggatgccagcgggacttccgcaacaagccttaccctgtcagggccaagatagaatacttcaacaacattctcacggtgctgttccacaacggcaacaccaacaacgacggtgactacgagatgtgcttccgtgcggagaacgtgttcctgccgaccaacggccactttggggtgtccgccgccacggggggcctggcagacgaccacgacgccctcaagttcctgacgacgagcctgcatgcggagggcacgcagccggccctggcccagggtatggccgactcagagaaggagaagttctccaaggagtatgaagtatacaaggacaagctggaaaagcagaaggaggagtaccggaagacgcacccggaggaggccgctaagcaggccatggagcacggccccgagcaggcctacgacacgcagcagcagcgcgagctgcgccagatcttcgagggccagagccacaaattgtttgaggggctcaaggcactgcaccgcaagctggacgaggtgctcgggcgccaggagcgcaccctgtcgctggtgtcggctggcggcgccggcgtggccgtgggcggtgttccgccaccgcagatgggtggagtgccgtcgctgcagaggcacgaagcagagtccctgctgagcagccagcgggagctgctgcagacggtggctcaggtcaagagctttgtggccgaggtgcatcaacgcacggccaccctgcaacaccagggggcgggaggcacccagggcctcacggccgagcagctgcaagtgctccaccaggtgcgggacagcgtggccagcatgcaccgggacgtctccaacaaccagccgcagaggactggctgcgcgacatcctgtctcagcactacccacttcttgctgtttgcaacgttgcagttggctgtcacgctgggctacttggtgtacaggagcagcaaagaggcggcggccaagaagttctactgagtgcagatctcgagccttgccttgccctcccctcccatggagtggaccttaaccccacagactgccagaaaccagtgttgccagaggagcccccctcccttcttattgggtggggtgccacagccatcacccattcttcgagacaaggccactgtttggggggaggggcaagagattcatccggggtgcgcaacaaaacatggccgtacagagggaggggtgctccagaactgggtcccagccacatcgttgcgtgggagcgcctttctncctcactctaaaaaaaaaaaaaaaaaaaaaaaa

SEQ ID NO: 16 - protein, 497 aa, Ixodes scapularis
MAAAVMNCLRTALLGALVVQLYATQIGHRKFEYKYSFKGPYLAQKDGSVPFWEYGGNCIASEEMVRITPSLKSKKGSIWSKLPTSFPWWEVELVFRTTGTGRIGADGLAFWYTDKKQAEGPVFGSSDKWTGLAIFFDSFDNDNKHNNPYIMGMVNDGTKAYDHESDGANQQLAGCQRDFRNKPYPVRAKIEYFNNILTVLFHNGNTNNDGDYEMCFRAENVFLPTNGHFGVSAATGGLADDHDALKFLTTSLHAEGTQPALAQGMADSEKEKFSKEYEVYKDKLEKQKEEYRKTHPEEAAKQAMEHGPEQAYDTQQQRELRQIFEGQSHKLFEGLKALHRKLDEVLGRQERTLSLVSAGGAGVAVGGVPPPQMGGVPSLQRHEAESLLSSQRELLQTVAQVKSFVAEVHQRTATLQHQGAGGTQGLTAEQLQVLHQVRDSVASMHRDVSNNQPQRTGCATSCLSTTHFLLFATLQLAVTLGYLVYRSSKEAAAKKFY

SEQ ID NO: 17 - DNA, 2475 bp, Ixodes scapularis
  misc_feature (1342)..(1342): n is a, c, g, or t
catcactagtagcgagacacgtgcgtaaaaatggggcccaaaacgctgtctaagcagcccgctaaagcttcttcatccacttccaagcgcaccgccggccccacaataagcaagcagacggaggacagcgatgacgaagggtcaagcagcgcctactccgacttggaggactccgaaggagccgacagcagcgactcgaacgatttgtcggacacggaggcgtcggaggatgactacgatgactcccaagacgaagaaaacacgaagattactttgactggggtggaggggaaggaccttgagttgagggggaaggaccaggaggcaccggtggagtctggcaaaaggtcggcatggcaccggcagcaagaggacgccaaggaggacagacgaacgcaagtggtggaagatgaatatgcctttgactcttccgacgaagaggacgttcgcaacacggttggcaacattcctctggagtggtacgagcactatccgcacatcggttatgatctggaaggcaagccaatcctgaagccgcctcgggttagtgacctggacgacttcctgaggaaaatggatgaccccaactattggaggacggtgaaggacaagagcacgggacaggacgttgtcctgaccgacgaagatgtggacctgattcagaggctgcagaaaggacagttccccagctcgacgactgacccttacgagccatttgaggacatcttttcgcacgagaccatgatccacccggtgaccaggcaccctccccagaaacgcagcttcgtgccttcaaggatagaaaaagcaatggtgtcaaagatggtgcacgcaatcaagatgggctggatcaagccccgagtaaagaagcatgacccagaaagattcagcctcctgtgggacaaggatgactcgacagcgggcagcaatgagcgaatgcagcgccacatcccggcacccaagatgaagctgccgggtcatgaggagtcttacaacccgccggccgaatacctcttcaccgaggaagaggaggccaagtggagagagcaggagcccgaagaacggcgcataaacttcctgcccgccaagtacccatgtctgcgcgcagtcccagcctacgaacgcttcattgaggagaggtttgagcgctgtctggatctctacttgtgcccgaggcagcggaagatgagggtgaatgtggatgcagaggacctgattcctcagctgcccaaacccaaggacctgcagcctttcccaagcattcagtctattgtctatgagggtcatacggactgtgtcctctgcctgtctttggagcctgcgggacagttctttgcatcanggtccgaggacggcaccgttcgcatttgggagctcttgacgggcangtgcctcaagaagttccagttcgaggcgcccgtgaagagcgtggcctggtgtccagttgtcgttcccatgaaactctgcgtggacaagactgtttccatgctggatgccggagttacggacaaactgctgccgttcaccacgggacaccgagttgtctgccctccccgaagagtcctcgggccaggcggcggtagtggagtgggagcagacgtcggcctcctctccagagttcctctcccggggggagcgtctgcgggtcgttcaccgccacggtgtggtgcaggtgacgtggcactcgaggggagactactttgccactgtcacggacgagggacaggccaccgtgcttgtccatcagttgtccacgcggcggttcgcaggctccccttcagcaaggcgaagggcggggtgtcccgggtgctgttccacccgctgcgccccttcctgctggtggcgtgccagcgcacagtgcgggtctaccacctgctcaagcaggagctggccaagaggctcacatccaattgcaagtggatctcgtgcatgggccgtccacccccaggtgacaatctgctgatcggcacgtacgagaagcggctgatgtggttcgatctggacctctcgaccaaaccgtaccagcagctgcgcatacacaatgccgccatccgcagtgtggcgttccatccgcgctatccactgtttgcgtccgccggcgacgatcgcagcgtgatcgtttcgcacggtatggtgtacaatgatttactgcaaaacccactgatcgtgccactgagacggctgaagaaccatgccatcagcaagggtatgggtgtgttggactgcgccttccatccccaccagccgtggatagtcacggccggagcagacagcacgctgcggctcttcacctaagccgggacgtcgtctggtgtacatagtgaatcgtcaagaccgtgccaataaaaggactccacacctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - protein, 754 aa, Ixodes scapularis
  misc_feature (438)..(438): Xaa can be any naturally occurring amino acid
MGPKTLSKQPAKASSSTSKRTAGPTISKQTEDSDDEGSSSAYSDLEDSEGADSSDSNDLSDTEASEDDYDDSQDEENTKITLTGVEGKDLELRGKDQEAPVESGKRSAWHRQQEDAKEDRRTQVVEDEYAFDSSDEEDVRNTVGNIPLEWYEHYPHIGYDLEGKPILKPPRVSDLDDFLRKMDDPNYWRTVKDKSTGQDVVLTDEDVDLIQRLQKGQFPSSTTDPYEPFEDIFSHETMIHPVTRHPPQKRSFVPSRIEKAMVSKMVHAIKMGWIKPRVKKHDPERFSLLWDKDDSTAGSNERMQRHIPAPKMKLPGHEESYNPPAEYLFTEEEEAKWREQEPEERRINFLPAKYPCLRAVPAYERFIEERFERCLDLYLCPRQRKMRVNVDAEDLIPQLPKPKDLQPFPSIQSIVYEGHTDCVLCLSLEPAGQFFASXSEDGTVRIWELLTGXCLKKFQFEAPVKSVAWCPVVVPMKLCVDKTVSMLDAGVTDKLLPFTTGHRVVCPPRRVLGPGGGSGVGADVGLLSRVPLPGGASAGRSPPRCGAGDVALEGRLLCHCHGRGTGHRACPSVVHAAVRRLPFSKAKGGVSRVLFHPLRPFLLVACQRTVRVYHLLKQELAKRLTSNCKWISCMGRPPPGDNLLIGTYEKRLMWFDLDLSTKPYQQLRIHNAAIRSVAFHPRYPLFASAGDDRSVIVSHGMVYNDLLQNPLIVPLRRLKNHAISKGMGVLDCAFHPHQPWIVTAGADSTLRLFT

SEQ ID NO: 19 - DNA, 447 bp, Ixodes scapularis
caaagatgctgctgttctgcccgacgtgcgccaacatcctcattgtggaacaaggcttggagtgcttccgtttcgcctgcaacacatgcccctacgtgcacaacatcaaggcgaagatgtcgaatcggaagtacccgcggctcaaggacgtggacgacgtgctcggcggtgcagccgcctgggagaatgttgactcgaccgaagagaagtgccccaagtgtggccatgagcgggcctattttatgcagatccagactaggtcggccgacgagcccatgaccaccttctacaagtgctgcaaccagctctgtggccaccagtggagggactgacagatggcggctttgacgaactcatgcccgtgcaaaatgcgtcggggggagagagttttggaataaaacatgcgccttactttcataaaaaaaaaaaaaaaaaaaaaaaaaaaaa

SEQ ID NO: 20 - protein, 108 aa, Ixodes scapularis
MLLFCPTCANILIVEQGLECFRFACNTCPYVHNIKAKMSNRKYPRLKDVDDVLGGAAAWENVDSTEEKCPKCGHERAYFMQIQTRSADEPMTTFYKCCNQLCGHQWRD

SEQ ID NO: 21 - DNA, 1567 bp, Ixodes scapularis
  misc_feature (785)..(785): n is a, c, g, or t
ccccccaggcgcagggcttcgttcaggtcgaccagggggccctccccgcaagccccgaggagcgccacctggcaagcatgcaggtcaatggatatgagaaccccacctacaagtacttcgaggccaacaccaactgagcggccacgcccccaggggagggggaaaagggggcggacggacgtattgtgcctgctgcgggctgcgggattagctcgtcccgcgttgttccgggagccagttggtttgcctcgcgtcttaggagtaggcacggcctcccttctgcacccggtcaaggaccatggttgttggggacacgagcggcgtggggcgcagccagcctgagctttgggtcccggtaccacggcaaaccgtttgttcccacccgcggaatgaaaattttgtttgcctcagtttctttcgaatcgagcgtcggcgccgcctccgacagccccgagtgcactctgtctgttgcgaaagaccaatggagtagttgacactcgggtcgcagctcgaacaagctcccgtaaaacgctacttaaccggggccggcgaccgagcgtagagcttgctgtgcgtagttgtggataaaactttttttttttgtgtgtgtgcttggtcacagacaatgggcagcttccgacgttagccacgcgccacacgctcgcctttgttttcttcttctcgcggttgtcatacttagtttccattggcgggttaacattccagtccgggcgggcgcccccgttcaggcgcgtcctgatcaaaattgagcatttggttgtgcngtgcatttattggccgcagcagggggttcccgggtgcacctggtgtcgtgacacgcatgtcgtgactttcccctcagacggttgtccttgctcatggctcgttcacacctctagtgctggtagtctctgttgcttaggtttgtaggagcacactacagcagagggtgtcacaaagttttctaagctgtatatacatgaggaaaacattgcgttgcacacacgcgagtttcggcctgtttttagttgggacagtgaacgttttttgtacaggttattatgtagtgcctacatttgtatgtgccagctgcatgtgttttcctgcatgtggggaagcctccgtgctgccccgagctgtgtgcggcccctcctgagtttccatgtgccatgtgcccagcctagggtgaactgggggtgcagatgcccttgcgcacggtgtgccccggcgagcattgtgtgtccgtaggccatcgacgctattcatgcgaaattaatgtggtcacagctgtcattgtctcagtgaacatatcatatgtccaaatttgtctcccctgtcagtgtgtgcttctcttggttctacacttgcctgcatttttgttagtttgccggactgtccttttcggtcccaggtcgacagcaggctataacaacaattccggtattttccagtatcgggtcacaccaggtgtaacctattgtgcatgtagtgtaacttgagtggaaaagctaaaataaaaatttgcaagagtctcactaaaaaaaaaaaaaaaaaaaaaaaaaaaaaa

SEQ ID NO: 22 - protein, 44 aa, Ixodes scapularis
PQAQGFVQVDQGALPASPEERHLASMQVNGYENPTYKYFEANTN

SEQ ID NO: 23 - DNA, 704 bp, Ixodes scapularis
  misc_feature (534)..(534): n is a, c, g, or t
tgagaagacactagaggacaagttcttcgagcatgaggtgatgctgaatgtgaatgcgttcatgcagcagttccattccggcgttttttatgcctacgtgaagctgaaggaacaagagtgccgcaacattgtctggattgccgaatgcgttgctcagcgtcatcggtccaagatcgataactacattccaatcttctagtcgctcgaggaaaagaaatgggccaattcggtagtttgtcggtgtaatatatatatatatatatatctacttcgcaaaattcttcagctagagtgtctatgtctggttagctgcgattgtgcgagaggggaaaaaaatgtagtcagtggcatgatcaaggaaggaaaaaaattggccaataacttttaccttttgaagttaaagcaagggttaaaataatgtctatttttacttcgctttaccgtgtgctggctattgctttgcaaacgttttttaaaatttttgcagttcgtctttcttcttttgagcacatatttattccagagttccaatanccttttatgtgtgaatgaatgactaatccatgttggggttggttaatggtgcattgttgaaaanataaaccccaactccagctggcctttggaaaaaaaaaaaaaaaaaaaaaaaaaaaaaaaaaaaaaaaaaaaaaaaaaaaaaaaaaaaaaaaaaaaaaaaaaaaaaa

SEQ ID NO: 24 - DNA, 681 bp, Ixodes scapularis
  misc_feature (432)..(432): n is a, c, g, or t
gtcacgggatttgggaagctgtcgtctgtcgtcctgcagtttcaaacggtttcaccaaaaacctttccgtctcgctgtcagacgccttgaaccatgactgagttctggctcatctcggctccgggcgagaaaacctgccaacagacttatgacaagctgctcagcgtcacaagcaacaagcagaacaacctctcgacctgctacaagttccaccttccggacttgaaggtgggtacgctggatcagttggttggcctctcggatgacttgggaaagctcgacacctatgtcgaaagcatcactcgaaaagtggccagctatctgggggacgtgcttgacgaccagagggacaaactagccgacaaccttccttgccaatggcttggggctggaggcctacctgaccccggttttcagtgggacatggccaantaccccatcaagcagttcgcctcaagagcatcacntgaantcatcagcnagcaagtgtctnanattngaccggtngaacctcnagnagcaagttancttgnttacaacaaccttnaanaacttaagnttcaantncatncgaaccccaaatccnccggggnaggccnggcnttnttccngttagccntggncntnaccttattgcgccaagggagccantttgtcnttgggggntcggganntacctta

SEQ ID NO: 25 - DNA, 720 bp, Ixodes scapularis
  misc_feature (488)..(488): n is a, c, g, or t
ctctcagcgactccgacgtccaaaagcagatcaagcacatgatggctttcatcgaccaggaagccaacgaaaaggcagaagaagtagacgccaaggcaggaagaagagttcaacatcgagaagggccgcctggtcacggagcaaaggctcaagatcatcgactactacacccgtcgagagaagcaagttgaactgcagcgcaagatccaaagctccaacatgctgaaccaggcccggctgaaggtgctgaaggcgggcgaggaccacattgcgacggtgctggaggaggccaagcgccgcctgggggacatcaccagggaccaggctcgctaccaagccctcctgcagagcatggttctgcaggcactgcttcagctcctcgagcaggaggtggtcgtccactgccgaccgcaagacgccgggctgctgaacttggacacgctgagtgccaagttcaaggaggccactggccgagaggtcaagctcantgtggagcccagcctggcttcgagcagctgcggcggagtcgagatgctctccaggcggggcaagattcgcgtctgcaacacgctcgagtcgcggctggacatgattgcccttcagctttctgccgcagatcaagacngncctnttcggcaggnacccccaaccgcaagttcatggactaggcgggctattgnccccgccattcnggccagtnagcttggaccgtgtttacng

SEQ ID NO: 26 - DNA, 212 bp, Dermacentor variabilis
atggcttgcgcaacattaaagcgaacacacgattgggatccgctgcatagtccaaacggaagatcgcccaaacgacggagatgtatgcctttgtcggtcacacaagcagcgactcccccaacaagggcacaccaaatcaacccttcacccttcggtgaagtgccaccgaaattaacttcagaggagatagcggccaacattcgggaggaaat

SEQ ID NO: 27 - protein, 70 aa, Dermacentor variabilis
MACATLKRTHDWDPLHSPNGRSPKRRRCMPLSVTQAATPPTRAHQINPSPFGEVPPKLTSEEIAANIREE

SEQ ID NO: 28 - DNA, 490 bp, Amblyomma americanum
atggcttgcgcaacattaaagcgaacgcacgactgggatcccctgcacagtccgaacggaagatcaccaaagcggcgaagatgtatgcctttttcagtcaacccaccagctcctccaacaagggcgcaccaaatcaatccctcgcctttcggcgacgtgccaccaaagctgacttcagaggagatagcagccaacatccgtgaggaaatgcggcggctgcagcggcgcaagcagctctgcttccagggtgcagagtgcagtagcccccccgaaggctgttcccccattggctccccctcccagcacaccggcggcctcctgtcacctgtgcgccgagaccagcccctgttcaccttccgccaagtgggactcatctgtgagcgcatgatgaaagagcgagagagccagatacgagaggagtatgaccacgtgctctccaccaagcttgcagagcagtacgacacatttgtcaagtttacgtacgaccaaa

SEQ ID NO: 29 - protein, 163 aa, Amblyomma americanum
MACATLKRTHDWDPLHSPNGRSPKRRRCMPFSVNPPAPPTRAHQINPSPFGDVPPKLTSEEIAANIREEMRRLQRRKQLCFQGAECSSPPEGCSPIGSPSQHTGGLLSPVRRDQPLFTFRQVGLICERMMKERESQIREEYDHVLSTKLAEQYDTFVKFTYDQ

SEQ ID NO: 30 - DNA, 442 bp, Boophilus microplus
atggcttgcgcaacattaaagcgaacacatgactgggaccccttgcacagtccgagtggcagatcgccgaagcgacgccgatgtatgcctctgtccccgcctcctacaagggcgcaccagatcgatccctcgcccttcggagacgtgccaccaaagttgacttcagaggagatagcagccaacatccgtgaggagatgcgacggctacaacggcgcaagcagctctgtttccagggcgctgaccctgaatcccagcataccagtggtctctcgtcaccagtgcatcgagaccagcccctgttcacgttccgccaggttgggctcatttgcgaacgaatgatgaaggagcgagagagcaagatacgggaggagtatgaccatgtgctatctaccaaactcgcagaacagtacgacacatttgtcaagtttacgtacgaccaaa

SEQ ID NO: 31 - protein, 147 aa, Boophilus microplus
MACATLKRTHDWDPLHSPSGRSPKRRRCMPLSPPPTRAHQIDPSPFGDVPPKLTSEEIAANIREEMRRLQRRKQLCFQGADPESQHTSGLSSPVHRDQPLFTFRQVGLICERMMKERESKIREEYDHVLSTKLAEQYDTFVKFTYDQ

SEQ ID NO: 32 - DNA, 23 bp, Artificial Sequence
  oligonucleotide primer
gcgtcgtgtggagcatcagcgac

SEQ ID NO: 33 - DNA, 23 bp, Artificial Sequence
  oligonucleotide primer
tcgcaacggacaacggcaggttg

SEQ ID NO: 34 - DNA, 23 bp, Artificial Sequence
  oligonucleotide primer
gcttgcgcaacattaaagcgaac

SEQ ID NO: 35 - DNA, 23 bp, Artificial Sequence
  oligonucleotide primer
tgcttgtttgcagatgcccatca

SEQ ID NO: 36 - DNA, 23 bp, Artificial Sequence
  oligonucleotide primer
gaaatatctgtgaaaccaaggcc

SEQ ID NO: 37 - DNA, 23 bp, Artificial Sequence
  oligonucleotide primer
attgcacaacacatcattaactg

SEQ ID NO: 38 - DNA, 486 bp, Rhipicephalus appendiculatus
atggcttgtgcgacattaaagcggacacatgactgggaccccttgcacagtccgagtggcagatcaccaaagcgacgccgatgcatgcctctgtccccgcctccaacgagggcgcaccagatcgatccctcgcccttcggagacgtgccaccaaagttgacttcagaggagatagcagccaacatccgcgaggagatgcgacggctacaacggcgcaagcagctctgttttcaaggcaccgacgccgaatcccagcataccagtggtctctcgtcaccggtgcgtcgagaccagcccctgttcacgttccgccaggttgggctcatttgcgaacgcatgatgaaggagcgagagagcaagatacgggaggagtatgaccacgtgctatccaccaagctcgcagaacagtacgacacgtttgttaaatttacctacgaccaaattcagaagcggtttgagggtgctacgcccagctatttgtcgtaa

SEQ ID NO: 39 - protein, 161 aa, Rhipicephalus appendiculatus
MACATLKRTHDWDPLHSPSGRSPKRRRCMPLSPPPTRAHQIDPSPFGDVPPKLTSEEIAANIREEMRRLQRRKQLCFQGTDAESQHTSGLSSPVRRDQPLFTFRQVGLICERMMKERESKIREEYDHVLSTKLAEQYDTFVKFTYDQIQKRFEGATPSYLS

(19) United States
(12) Patent Application Publication
(10) Pub. No.: US 2005/0123554 A1
(43) Pub. Date: Jun. 9, 2005

(54) Protective antigens and vaccines for the control of multi species tick infestations

(75) Inventors: Jose de Jesus de La Fuente, Stillwater, OK (US); Katherine M. Kocan, Perkins, OK (US); Consuelo Garcia-Almazan, Stillwater, OK (US); Edmour F. Blouin, Perkins, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(73) Assignee: The Board of Regents for Oklahoma State University

(21) Appl. No.: 10/972,789

(22) Filed: Oct. 25, 2004

Related U.S. Application Data
(63) Continuation-in-part of application No. 10/425,563, filed on Apr. 29, 2003.
(60) Provisional application No. 60/376,251, filed on Apr. 29, 2002.

(51) Int. Cl. C12Q 1/68; C07H 21/04

(52) U.S. Cl. 424/185.1; 530/350; 435/6; 435/69.3; 435/348; 536/23.5

(57) ABSTRACT

Broad range protective antigens against tick infestations, gene sequences and encoded proteins for such antigens, related vaccines and methods useful to induce an immune response.

### CROSS REFERENCE TO RELATED APPLICATIONS

This application is a continuation-in-part of copending U.S. patent application Ser. No. 10/425,563, filed Apr. 29, 2003, which application claims the benefit of U.S. Provisional Patent Application Ser. No. 60/376,251 filed Apr. 29, 2002. Both noted priority applications are incorporated herein by reference.

### BACKGROUND OF THE INVENTION

### TECHNICAL FIELD

The present invention relates to the identification of protective antigens against tick infestations, gene sequences and encoded proteins for such antigens, related vaccines and methods useful to induce an immune response in mammals, which are protective against a broad range of tick species.

### BACKGROUND

Ticks parasitize wild, domesticated animals and humans and transmit pathogens including fungi, bacteria, viruses and protozoon. Currently, ticks are considered to be second in the world to mosquitoes as vectors of human diseases, but they are considered to be the most important vector of pathogens in North America (Parola and Raoult, 2001). Ixodes spp. are distributed worldwide and act as vectors of human diseases caused by Borrelia burgdorferi (Lyme disease), Anaplasma phagocytophila (human granulocytic ehrlichiosis), Coxiella burnetti (Q fever), Francisella tularensis (tularemia), B. afzelii, B. lusitaniae, B. valaisiana and B. garinii, Rickettsia helvetica, R. japonica and R. australis, Babesia divergens and tick-borne encephalitis (TBE) and Omsk Hemorrhagic fever viruses (Estrada-Peña and Jongejan, 1999; Parola and Raoult, 2001). Throughout eastern and southeastern United States and Canada, I. scapularis (the black legged tick) is the main vector of B. burgdorferi sensu stricto and A. phagocytophila (Estrada-Pefia and Jongejan, 1999; Parola and Raoult, 2001). Besides Ixodes, other tick species of medical and veterinary importance include those of the genera Amblyomma, Dermacentor, Rhipicephalus and Boophilus.

Control of tick infestations is difficult and often impractical for multi-host ticks such as Ixodes spp. Presently, tick control is effected by integrated pest management in which different control methods are adapted to one area or against one tick species with due consideration to their environmental effects. Control of ticks by vaccination, however, would avoid environmental contamination and selection of drug resistant ticks that result from repeated acaricide application (de la Fuente et al., 1998; Garcia-Garcia et al., 1999, de la Fuente and Kocan 2004). Anti-tick vaccines would also allow for inclusion of multiple antigens in order to target a broad range of tick species and for incorporation of pathogen-blocking antigens (de la Fuente and Kocan 2004).

Recently, vaccines have been developed against one-host Boophilus spp. (Willadsen, 1997; Willadsen and Jongejan, 1999; de la Fuente et al., 1999; 2000; de Vos et al., 2001; de la Fuente and Kocan 2004). The recombinant B. microplus BM86 gut antigen included in commercial vaccine formulations TickGARD (Hoechst Animal Health, Australia) and Gavac (Heber Biotec S. A., Havana, Cuba) also confers partial protection against phylogenetically related Hyalomma and Rhipicephalus tick genera (de la Fuente et al., 2000; de Vos et al., 2001; de la Fuente and Kocan 2004). However, immunization with BM86 failed to protect against the more phylogenetically distant Amblyomma spp. (de Vos et al., 2001). These results suggest that using Bm86 or a closely related gene for the production of vaccines against Ixodes spp. or other tick genera phylogenetically distant from Boophilus spp. (Black and Piesman, 1994) could be impractical. Therefore, there remains a need to identify broad range vaccine candidates against tick infestations across phylogenetically distant species (de la Fuente and Kocan 2004).

In parent application Ser. No. 10/425,563 we described the identification by ELI and sequence analysis of protective cDNA clones against experimental infestations with I. Scapularis in what was the first example of the application of ELI to arthropods and particularly to ticks (Almazan et al 2003 a,b). It was thought that these protective antigens, although identified for I. scapularis, might be cross protective between Ixodes species considering the high degree of conservation of gene sequences and protein function between species of the same genus.

Surprisingly, it has been discovered that certain of the protective antigens exhibit activity and are expressed across tick species, and in some circumstances across genera, thus making possible the use of these common antigens in broad range anti-tick vaccine formulations.

### SUMMARY OF THE INVENTION

In the parent application, numerous protective antigens derived from I. scapularis were identified and shown to possess protective properties against I. scapularis tick infestations. Among these antigens were clones and associated polypeptides designated 4D8, 4F8 and 4E6. Herein the activity of these particular protective antigens is demonstrated to extend to other tick species, and, in the case of 4D8, 4F8 and 4E6 to non-Ixodes tick species. In addition, there is provided the recently discovered sequences (DNA and protein) of tick protective antigen 4D8 derived from genus Rhipicephalus, particularly Rhipicephalus appendiculatus.

Sequence conservation and expression of 4F8, 4D8 and 4E6 were analyzed in I. scapularis related species, I. pacificus and I. ricinus, and in D. variabilis, R. sanguineus, B. microplus and A. americanum by RT-PCR using the primers specific for the known I. scapularis sequences. Expression was detected in I. ricinus for all three genes and in I. pacificus for 4D8 and 4E6. Expression of 4D8 was also detected in D. variabilis, B. microplus, R. sanguineus and A. americanum. Corresponding 4D8 genes of D. variabilis, A. americanum and B. microplus were cloned and sequenced.

The detection of 4D8 and 4E6 expression in non-Ixodes tick species were the basis for an experiment in which rabbits were immunized with I. scapularis derived 4F8, 4D8 and 4E6 and challenged with I. scapularis, D. variabilis and A. americanum nymphs. Results support using the inventive protective antigens in vaccine formulations for the control of multiple tick species.

Accordingly, in one embodiment of the present invention there is provided cDNA sequences, protein encoding fragments thereof, and derived protein sequences for the multi-species protective antigens denominated 4F8, 4D8 and 4E6.

In another embodiment of the present invention there is provided a vaccine composition comprising the inventive protective recombinant proteins and/or modified cDNAs separately or which may optionally be combined with adjuvant to enhance the protection efficacy of vaccine preparations against multiple tick species, wherein the vaccine composition further comprises a pharmaceutically acceptable carrier or diluent. The vaccine composition also may optionally be combined with tick-borne pathogen components to provide a means to control tick-borne infections, wherein the vaccine composition further comprises a pharmaceutically acceptable carrier or diluent and adjuvant.

In another embodiment of the present invention there is provided a method for inducing an immune response in a mammal to provide immune protection, which reduces or affects infestations by target ticks and/or transmission of tick-borne pathogens, the method comprising administering to at-risk human population and mammalian reservoir an effective amount of a vaccine composition comprising the inventive protective recombinant proteins and/or modified cDNAs alone or in combination with an adjuvant or tick-borne pathogen components to provide a means to control tick infestations and to reduce transmission to humans of tick-borne infections, wherein the vaccine composition further comprises a pharmaceutically acceptable carrier or diluent.

A better understanding of the present invention and its objects and advantages will become apparent to those skilled in this art from the following detailed description, wherein there is described only the preferred embodiment of the invention, simply by way of illustration of the best mode contemplated for carrying out the invention. As will be realized, the invention is capable of modifications in various obvious respects, all without departing from the scope and spirit of the invention. Accordingly, the description should be regarded as illustrative in nature and not as restrictive.

### BRIEF DESCRIPTION OF THE DRAWINGS

FIG. 1 is a summary of the cDNA ELI approach used to identify protective antigens against I. scapularis infestations.

FIG. 2A is a graph depicting the results of a primary screen of cDNA pools (A-H 1-4, A5) by ELI. V, control mice injected with 1 μg vector DNA alone. *α<0.1, **α<0.05 (Tukey's post-hoc test for pair comparisons after ANOVA). Number in boxes represent values for inhibition of tick infestation with respect to the control group.

FIG. 2B is a graph depicting the results of a primary screen of cDNA pools (A6-A10, B-H 5-8) by ELI. V, control mice injected with 1 μg vector DNA alone. *α<0.01, **α<0.05 (Tukey's post-hoc test for pair comparisons after ANOVA). Number in boxes represent values for inhibition of tick infestation with respect to the control group.

FIG. 3 is a graph depicting the results of a tertiary screen by ELI of cDNA sub-pools formed according to the predicted function of encoded proteins. Only groups with 1≦15% are shown (white bars). The number of engorged larvae per mouse is expressed as mean±SD (black bars). Control mice were injected with mitochondrial (MT) cDNAs. *P≦0.05 (Student's t-test).

### DETAILED DESCRIPTION OF THE INVENTION

Before explaining the present invention in detail, it is important to understand that the invention is not limited in its application to the details of the construction illustrated and the steps described herein. The invention is capable of other embodiments and of being practiced or carried out in a variety of ways. It is to be understood that the phraseology and terminology employed herein is for the purpose of description and not of limitation.

In the parent application, there were provided 25 separate and distinct sequences comprising 14 cloned cDNA molecules and 11 deduced amino acid sequences of encoded polypeptides, said sequences having been isolated and identified as protective against I. scapularis in accordance with the following described experimental methodology.

### EXAMPLE 1

### Construction of an I. scapularis cDNA Library and Screening for Protective Antigens by ELI

Tick Cells

Monolayers of IDE8 (ATCC CRL 1973) cells, originally derived from embryonic I. scapularis, were maintained at 31° C. in L-15B medium supplemented with 5% foetal bovine serum, tryptose phosphate broth and bovine lipoprotein concentrate after Munderloh et al. (1994). Cells were subcultured at 1:5-1:10 when monolayers reached a density of approximately 10⁷ cells/T-25 flask. Medium was replaced weekly.

Library Construction

A cDNA expression library was constructed in the vector pEXPI containing the strong cytomegalovirus CMV_{1E} promoter (Clontech). Because we planned to target the early larval stages of I. scapularis, we chose to construct our library from cultured embryonic I. scapularis IDE8 cells-derived poly(A)+ RNA. The cDNA library contained 4.4×10⁶ independent clones and a titer of approximately 10₁₀ cfu/ml with more than 93% of the clones with cDNA inserts. The average cDNA size was 1.7 kb (0.5-4.0 kb).

Primary Screen

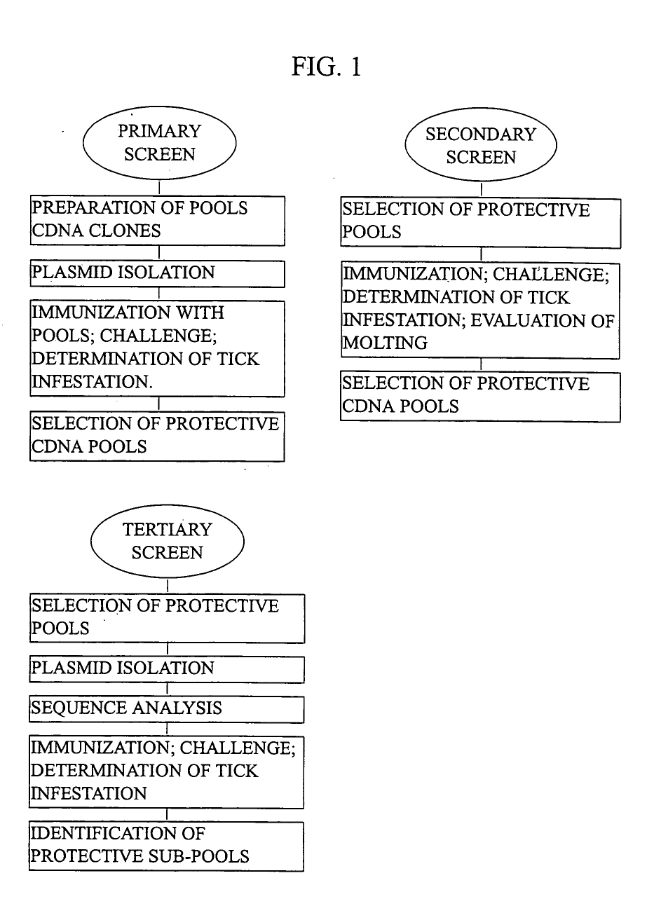

The overall schema for identification of protective antigens through ELI, sequential fractionation and sequence analysis is shown in FIG. 1.

Ninety-six LBA (master) plates containing an average of 41 (30-61) cDNA clones per plate were prepared. Replicas were made and clones from each plate were pooled, inoculated in Luria-Bertani with 50 μg/ml ampicillin, grown for 2 hr in a 96 wells plate and plasmid DNA purified from each pool (Wizard SV 96 plasmid DNA purification system, Promega, Madison, Wis., USA). BALB/c female mice, 5-6 weeks of age at the time of first vaccination, were used. Mice were cared for in accordance with standards set in the Guide for Care and Use of Laboratory Animals. Mice were injected with a 1 ml tuberculin syringe and a 27 gauge needle at days 0 and 14. Three mice per group were each immunized IM in the thigh with 1 μg DNA/dose in 50 μl PBS. Two groups of 3 mice each were included as controls. One group was injected with 1 μg vector DNA alone and the second with saline only. Two weeks after the last immunization, mice were infested with 100 I. scapularis larvae per mouse. Ticks were artificially reared at the Oklahoma State University tick rearing facility by feeding larvae on mice, nymphs on rabbits and adults on sheep and using for infestation in our experiments the larvae obtained from the eggs oviposited by a single female. Twelve hours after tick infestation, larvae that did not attach were counted to calculate the number of attached larvae per mouse and mice were transferred to new cages. Replete larvae dropping from each mouse were collected daily and counted during 7 days. The inhibition of tick infestation (I) for each test group was calculated with respect to vector-immunized controls as [1−(<RL>n/<RL>c×<RL>ic/<RL>in)]×100, where <RL>n is the average number of replete larvae recovered per mouse for each test group, <RL>c is the average number of replete larvae recovered per mouse for control group, <RL>ic is the average number of larvae attached per mouse for control group, and <RL>in is the average number of larvae attached per mouse for each test group.

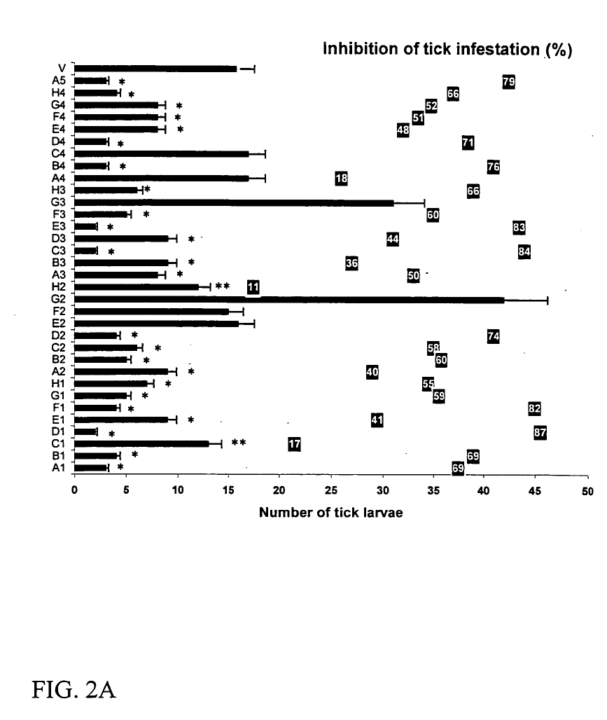

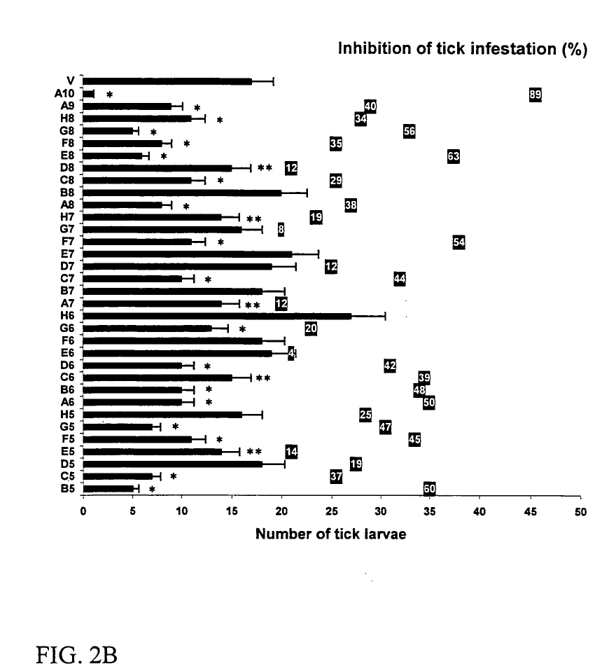

Pools of 41 (30-61) I. scapularis cDNA clones were screened by ELI. Only 33 cDNA pools and controls were analyzed per experiment. The average tick infestation level was 50±13 and 56±15 and 56±15 and 54±18 larvae/mouse for cDNA immunized and control mice, respectively (P>0.05) (Table 1). The average number of engorged larvae recovered per mouse was 9±3 and 13±4 in the cDNA-immunized mice and 16±4 and 17±3 in the control vector-immunized group (P<0.05) (Table 1). No reduction was observed in the number of larvae collected from mice that received the vector DNA compared to saline-immunized controls. The maximum number of engorged larvae was collected 3 to 4 days after infestation. However, in mice immunized with cDNA pools B5, A8 and A10 (FIG. 2) a retardation of larval development in 1 to 2 days was recorded. The average inhibition of tick infestation (I) was 49±28% and 30±22% (Table 1). After two experiments covering the analysis of 66 pools (2705 clones), 9 protective pools (351 clones) were selected producing an inhibition of tick infestation 1≧60% (FIGS. 2A and 2B and Table 1). When we started these experiments, we planed to screen over 4000 cDNA clones considering the complexity of the tick genome. However, to our surprise 9 protective cDNA pools were identified after screening 66 pools containing 2705 cDNA clones. This result probably reflects the possibility of interfering with tick infestations at many different levels that involve a Pleiades of gene products. Results from vaccination experiments against ticks employing recombinant antigens support this view (reviewed by Mulenga et al., 2000). Because of the complexity of the screening procedure in mice vaccinated and challenged with tick larvae, it was difficult to work with more than 9 protective cDNA pools. Therefore we did not continue screening new cDNA pools and focused our attention on the 9 pools selected after the primary screen.

Secondary Screen

The secondary screen was done to verify the protective capacity of the cDNA pools selected after the primary screen (FIGS. 2A and 2B). After the primary screen of 66 cDNA pools (2705 clones), 9 pools with 1>60% were selected for the secondary screen (re-screening) employing 5 mice per group as described above. Engorged larvae were kept for molting in a 95% humidity atmosphere. Molting of engorged larvae was evaluated by visual examination of tick nymphs under a stereomicroscope 34 days after last larval collection. The inhibition of molting (M) for each test group was calculated with respect to vector-immunized controls as [1−(MLn/MLc×RLc/RLn)]×100, where MLn is the number of nymphs for each test group, MLc is the number of nymphs for the control group, RLc is the number of larvae recovered for the control group, and RLi is the number of larvae recovered for each test group. Control mice were immunized with the negative (I=0%) F2 cDNA pool or saline only. A group was included immunized SC with two doses of 100 μg of total IDE8 tick cell proteins per dose in Freund's incomplete adjuvant.

All 9 protective cDNA pools gave positive results in the secondary screen (data not shown). The tick infestation levels were higher in this experiment (average 85±6 and 84±3 larvae/mouse for cDNA-immunized and control mice, respectively; P>0.05). Nevertheless, the average number of engorged larvae recovered per mouse was 39±7 and 26±6 for control and cDNA-immunized mice, respectively (P<0.05). The group immunized with total IDE8 tick cell proteins was protected with 1=33%. Again, no reduction was observed in the number of larvae collected from mice that received the control cDNA (F2 negative pool after the primary screen; FIG. 2A) compared to saline-immunized controls.

In the secondary screen, molting of engorged larvae was evaluated after 34 days. Molting was affected in all but one test cDNA-immunized group. Inhibition of molting in test cDNA-immunized mice compared to the control cDNA-immunized group varied from 0% to 12% (6±4%). The inhibition of molting was higher than 50% only in the larvae collected from mice immunized with cDNA pools B5 and A10, which showed a retardation of larval development in 1 to 2 days as in the primary screen. No differences were observed between control cDNA and saline-immunized mice. Among the larvae that did not molt to nymph, some were visibly damaged and presented a strong red coloration. The percent of red larvae in cDNA-immunized mice varied between 3% to 18% (7±5%) while in the saline and control cDNA-immunized groups red larvae represented the 6% and 4%, respectively.

Tertiary Screen

For the tertiary screen, 64 clones were grouped in 16 sub-pools each containing 1 to 17 plasmids according to the predicted function of encoded proteins (e.g., all the plasmids that encoded histone proteins were grouped together) and used with 4 sub-pools containing 182 clones of unknown function or with sequences without homology to sequence databases to immunize 4 mice per group. Mice were immunized with 0.3 μg/plasmid/dose in 50 μl PBS and evaluated as described above. Control mice were immunized with a pool of 20 plasmids containing mitochondrial cDNAs.

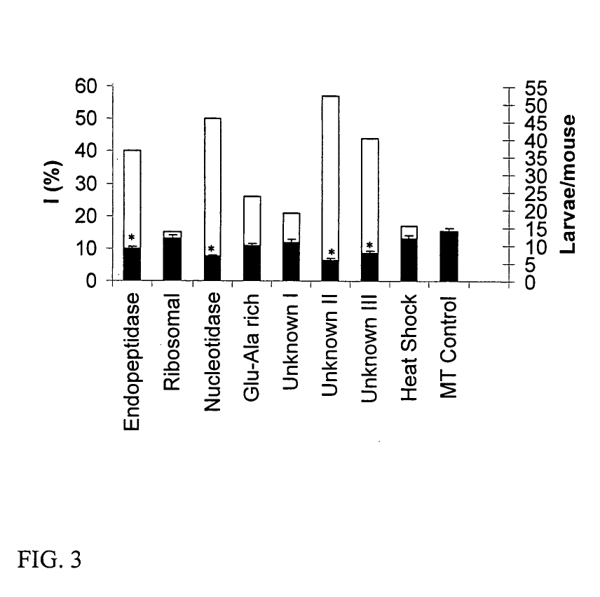

Tick infestation levels were similar in all test groups (72±2 larvae/mouse) and in control mice (69±2 larvae/mouse) (P>0.05). The number of engorged larvae recovered per mouse was also similar between test (16±7) and control (14±6) mice (P>0.05). However, the groups immunized with cDNA sub-pools containing clones with putative endopeptidase, nucleotidase, ribosomal proteins, heat shock proteins, glutamine-alanine-rich proteins and 3 of the sub-pools with unknown function or with sequences without homology to sequence databases had I≧15% (FIG. 3). Furthermore, among them, the groups immunized with sub-pools containing clones with a putative endopeptidase, nucleotidase and two of the cDNA sub-pools with unknown function or with sequences without homology to sequence databases resulted in lower infestation levels compared to control mice (P≦0.05) and I≧40% (FIG. 3). Clones homologous to chorion proteins, vitellogenin receptors, and peptidoglycan recognition proteins were selected for they potential protection capacity in other stages of tick development.

Statistical Analysis

The number of larvae attached per mouse and the number of engorged larvae recovered per mouse 7 days after infestation were compared by Analysis of Variance (ANOVA) followed by a series of Tukey's post-hoc tests for pair comparisons between cDNA-immunized and control vector DNA-immunized mice (primary screen), and by Student's t-test between mice immunized with positive cDNA pools and the control negative F2 cDNA pool (secondary screen) or between test cDNA sub-pools-immunized and control mice immunized with mitochondrial cDNAs (tertiary screen).

### EXAMPLE 2

### Sequence Analysis of Protective Clones

All the 351 cDNA clones in the 9 pools that resulted positive in the secondary screen were sequenced. DNA from individual clones in these pools was purified (Wizard SV 96 plasmid DNA purification system, Promega) from the master plate and partially sequenced. In most cases a sequence larger than 700 nucleotides was obtained. Nucleotide sequences were analyzed using the program AlignX (Vector NTI Suite V 5.5, InforMax, North Bethesda, Md., USA). BLAST (Altschul et al., 1990) was used to search the NCBI databases to identify previously cloned sequences that may have homology to those that we sequenced. Sequence analysis allowed grouping the clones according to sequence identity to DNA databases and predicted protein function. The protective clones selected after the tertiary screen were fully sequenced.

Comparison to sequence databases permitted to identify sequence identity to previously reported genes with known function in 152 (43%) of the clones (Table 2). Fifty seven percent of the sequences were homologous to genes with unknown function or had no significant identity to previously reported sequences (Table 2). Of the clones with sequence identity to genes with known function, 85% were homologous to arthropod sequences. Ninety-three clones (61%) contained sequences homologous to Drosophila melanogaster, 5 (3%) to other insects and 32 (21%) to Ixodid tick species. Thirty percent of the clones were eliminated from further analysis based on their sequence identity, including those containing similar sequences (Table 2). The protective clones included antigens homologous to endopeptidases, nucleotidases, chorion proteins, vitellogenin receptors, peptidoglycan recognition proteins, glutamine-alanine rich proteins, ribosomal proteins, and heat-shock proteins.

Summary of Results

The results obtained with the various protective clones identified in the Sequence Listing, along with certain selected expressed proteins, are summarized in Table 4.

SEQ ID NO:1 denotes the clone designated 4E6, wherein the relevant protein encoding fragment has been identified as comprising residues 1-117, which encodes the polypeptide shown in SEQ ID NO: 2.

SEQ ID NO:3 denotes the clone designated 4D8, wherein the relevant protein encoding fragment has been identified as comprising residues 80-575, which encodes the polypeptide shown in SEQ ID NO: 4.

SEQ ID NO:5 denotes the clone designated 4F8, wherein the relevant protein encoding fragment has been identified as comprising residues 1-951, which encodes the polypeptide shown in SEQ ID NO: 6.

SEQ ID NO:7 denotes the clone designated 4G11, wherein the relevant protein encoding fragment has been identified as comprising residues 1-697, which encodes the polypeptide shown in SEQ ID NO: 8.

SEQ ID NO:9 denotes the clone designated 4D6, wherein the relevant protein encoding fragment has been identified as comprising residues 198-1025, which encodes the polypeptide shown in SEQ ID NO: 10.

SEQ ID NO:11 denotes the clone designated 3E1, wherein the relevant protein encoding fragment has been identified as comprising residues 3-578, which encodes the polypeptide shown in SEQ ID NO: 12.

SEQ ID NO:13 denotes the clone designated 1C10, wherein the relevant protein encoding fragment has been identified as comprising residues 1-1119, which encodes the polypeptide shown in SEQ ID NO: 14.

SEQ ID NO:15 denotes the clone designated 3E10, wherein the relevant protein encoding fragment has been identified as comprising residues 51-1544, which encodes the polypeptide shown in SEQ ID NO: 16.

SEQ ID NO:17 denotes the clone designated 4F11, wherein the relevant protein encoding fragment has been identified as comprising residues 31-2295, which encodes the polypeptide shown in SEQ ID NO: 18.

SEQ ID NO:19 denotes the clone designated 3C12, wherein the relevant protein encoding fragment has been identified as comprising residues 6-332, which encodes the polypeptide shown in SEQ ID NO: 20.

SEQ ID NO:21 denotes the clone designated 2C12, wherein the relevant protein encoding fragment has been identified as comprising residues 3-137, which encodes the polypeptide shown in SEQ ID NO: 22.

SEQ ID NOS: 22, 23 AND 24, denote, respectively, clones 1A9, 1B2 and 4A4, each comprising a partial sequence with no associated polypeptide.

It has now been discovered that certain of the protective antigens exhibit activity and are expressed across tick species, and in some circumstances across genera, thus making possible the use of these common antigens in broad range anti-tick vaccine formulations.

### EXAMPLE 3

### Extension of I. scapularis Protective Antigens to Other Tick Species

Methods

RNA Extraction and Reverse Transcription (RT)-PCR

Total RNA was extracted from guts and salivary glands dissected from 30 unfed I. scapularis females, approximately 100 and 1000 I. scapularis nymphs and larvae, respectively, the egg mass oviposited by one I. scapularis female, salivary glands dissected from 10 D. variabilis males, salivary glands and guts dissected from 20 A. americanum and R. sanguineus adults, 10 B. microplus adults and 10 I. pacificus females, respectively and 100-150 I. ricinus larvae. Total RNA was extracted from homogenized tick samples using TR1 Reagent (Sigma), except for I. ricinus and B. microplus RNA which were extracted using the RNA Instapur kit (Eurogentec, Seraing, Belgium) and the RNeasy mini kit (Qiagen, Valencia, Calif., USA), respectively, according to the manufacturer's instructions. The final RNA pellet was resuspended in 50-100 μl diethyl pyrocarbonate-treated distilled deionized sterile water. RT-PCR reactions were performed using the Access RT-PCR system (Promega, Madison, Wis., USA). One μl RNA was reverse transcribed in a 50 μl reaction mixture (1.5 mM MgSO₄, 1× avian myeloblastosis virus (AMV) RT/Thermus flavus (Tfl) reaction buffer, 10 mM random hexamer, 0.2 mM each deoxynucleoside triphosphate (dNTP), 5 U AMV RT, 5u Tfl DNA polymerase (Promega), 10 pmol of each primer) at 48° C. for 45 min. After 2 min incubation at 94° C., PCR was performed in the same reaction mixture with specific primers and amplification conditions (Table 5). Control reactions were performed using the same procedures but without RT to control for DNA contamination in the RNA preparations and without RNA added to control contamination of the PCR reaction. Positive control reactions for the PCR were performed with plasmid DNA containing the cloned tick cDNAs and with genomic DNA extracted from I. scapularis IDE8 cells. PCR products were electrophoresed on 1% agarose gels to check the size of amplified fragments by comparison to a DNA molecular weight marker (1 Kb Plus DNA Ladder, Promega).

Cloning and Sequencing of D. variabilis, A. americanum and B. microplus 4D8

Fragments of the D. variabilis, A. americanum and B. microplus 4D8 cDNA were amplified by RT-PCR and cloned into pGEM-T (Promega). Four clones of each were sequenced at the Core Sequencing Facility, Department of Biochemistry and Molecular Biology, Noble Research Center, Oklahoma State University.

Vaccination of Rabbits and Challenge with I. scapularis, D. variabilis and A. americanum Nymphs

An experiment was designed to evaluate the effect of tick protective antigens on nymphal stages of I. scapularis, D. variabilis and A. americanum. One New Zealand White rabbit per group was each immunized with 3 doses (weeks 0, 4 and 7) containing 50 μg/dose purified 4F8 and 4D8 proteins or 4E6 synthetic peptide derived from I. scapularis, a combination of 4F8+4D8+4E6 or bovine serum albumin (Sigma) as control in FIA (Sigma). Animals were cared for in accordance with standards specified in the Guide for Care and Use of Laboratory Animals. Rabbits were injected subcutaneously with 500 μI/dose using a 1 ml tuberculin syringe and a 27½G needle. Two weeks after the last immunization, rabbits were infested with 100 I. scapularis nymphs per rabbit in one ear and 110 nymphs of each D. variabilis and A. americanum on the other ear. The number of nymphs attached was recorded and engorged nymphs dropping from each rabbit's ear were collected daily and counted during 7 days. D. variabilis and A. americanum engorged nymphs were separated under a dissecting light microscope. Engorged nymphs collected after completion of the feeding period were counted and weighted. The inhibition of tick nymphal infestation was calculated with respect to the control group as described previously for larval tick infestations (Almazan et al., 2003). The reduction on the weight of engorged nymphs was determined in experimental groups with respect to the nymphs collected from the control group.

Results

Expression of Protective cDNAs

Expression of genes encoding for tick protective antigens was analyzed at mRNA and protein levels by RT-PCR and immunohistochemistry, respectively. Expression of 4F8, 4D8 and 4E6 mRNA was detected in all I. scapularis developmental stages and in guts and salivary glands from adult ticks (Table 6). Control reactions ruled out contamination with genomic DNA or during the PCR. Furthermore, PCR of tick genomic DNA showed that the size of the amplified DNA fragments was higher than the size corresponding to cDNA fragments, probably due to the presence of intron sequences in the analyzed genes (Table 6).

Sequence conservation and expression of 4F8, 4D8 and 4E6 were analyzed in I. scapularis related species, I. pacificus and I. ricinus, and in D. variabilis, R. sanguineus, B. microplus and A. americanum by RT-PCR (Table 6) using the primers derived from I. scapularis sequences (Table 5). Expression was detected in I. ricinus for all three genes and in I. pacificus for 4D8 and 4E6. Expression of 4D8 was also detected in D. variabilis, B. microplus, R. sanguineus and A. americanum.

Sequence of D. variabilis, A. americanum and B. microplus 4D8

The sequences of D. variabilis, A. americanum and B. microplus 4D8 cDNAs and their deduced amino acid sequences are provided in SEQ ID NOS: 26-31. The sequences associated with tick protective antigen 4D8 derived from Rhipicephalus appendiculatus are provide in SEQ ID NOS: 38-39.

Protective Properties of Tick Antigens Against I. scapularis, D. variabilis and A. americanum Nymphal Infestations

The detection of 4D8 and 4E6 expression in non-Ixodes tick species were the basis for an experiment in which rabbits were immunized with I. scapularis derived protective antigens and challenged with I. scapularis, D. variabilis and A. americanum larvae. The results suggested an effect of 4D8 immunization on the inhibition of nymphal infestations in all three tick species and a notable effect on D. variabilis nymphs reflected in the number of visibly damaged ticks and the reduction in the weight of engorged nymphs (Table 7). Immunization with 4F8 had an effect on I. scapularis nymphal infestations only and the effect of 4E6 immunization was reflected on A. americanum infestations and on the weight of D. variabilis engorged nymphs (Table 7). The synergistic effect of the immunization with all three antigens was observed for 4D8 in I. scapularis nymphs (Table 7).

Experiment 3, then, demonstrates the surprising utility of using I. scapularis tick protective antigens 4D8, 4F8 and 4E6 in vaccine formulations for the control of multiple tick species.

Thus, the present invention relates to the identification of these antigens which are protective against a broad range of tick species, gene sequences and encoded proteins for such antigens, related vaccines and methods useful to induce an immune response. More generally, the invention concerns the given cDNA sequences and any nucleotide sequence coding for a protein which is capable of eliciting an antibody or other immune response (e.g., T-cell response of the immune system) which recognizes an epitope(s) of the amino acid sequences depicted in the Sequence Listing, including less than the full cDNA sequences and mutants thereof. Hence the nucleotide sequence may encode a protein which is the entire antigen encoded by the variously identified bases, or a fragment or derivative of the antigen or a fusion product of the antigen or fragment and another protein, provided that the protein which is produced from such sequence is capable of eliciting an antibody or other immune response which recognizes an epitope(s) of the given amino acid sequences.

As a result, the invention encompasses DNA sequences which encode for and/or express in appropriate transformed cells, proteins which may be the full length antigen, antigen fragment, antigen derivative or a fusion product of such antigen, antigen fragment or antigen derivative with another protein.

As will be appreciated by those of ordinary skill in the art, a nucleotide sequence encoding the inventive antigens or variants thereof may differ from the native sequences presented herein due to codon degeneracies, nucleotide polymorphisms, or nucleotide substitutions, deletions or insertions. While several embodiments of such molecules are depicted in the Sequence Listing, it should be understood that within the context of the present invention, reference to one or more of these molecules includes variants that are naturally occurring and/or synthetic sequences which are substantially similar to the sequences provided herein and, where appropriate, the protein (including peptides and polypeptides) that are encoded by these sequences and their variants. As used herein, the nucleotide sequence is deemed to be “substantially similar” if: (a) the nucleotide sequence is derived from the coding region of a native gene of the various noted tick species and encodes a peptide or polypeptide possessing the described antigenicity (including, for example, portions of the sequence or allelic variations of the provided sequences); or (b) the nucleotide sequences are degenerate (i.e., sequences which encode the same amino acid using a different codon sequence) as a result of the genetic code to the nucleotide sequences defined in (a); or (c) the nucleotide sequence is at least 95% identical to a nucleotide sequence provide herein, or (d) is a complement of any of the sequences described in (a-c).

Proteins included within the present invention have an amino acid sequence depicted in the Sequence Listing and homologs thereof. Other included proteins consist of a fragment of said sequence capable of eliciting an antibody or other immune response which recognizes an epitope(s) of the amino acid sequences depicted and a mutuant of said sequence capable of eliciting an antibody or other immune response which recognizes an epitope(s) of such amino acid sequences. With respect to homology to the native sequence, a variant should preferably have at least 95% amino acid sequence homology. As used herein, amino acid “homology” is determined by a computer algorithm incorporated in a protein database search program commonly used in the art, for example, as incorporated in the programs BLAST (BLAST.™., a computer program) (Altschul et al., Nucleic Acids Res. (25) 3389-3402, 1997) or DNA STAR MEGALIGN (DNA STAR MEGALIGN.™., a computer program) which return similar results in homology calculations.

The nucleotide sequences may be inserted into any of a wide variety of expression vectors by a variety of procedures. Such procedures and others are deemed to be known by those skilled in the art. Suitable vectors include chromosomal, nonchromosomal and synthetic DNA sequences; e.g., derivatives of SV40; bacterial plasmids; phage DNAs; yeast plasmids; vectors derived from combinations of plasmids and phage DNAs, viral DNA such as baculovirus, vaccinia, adenovirus, fowl pox virus, pseudorabies, etc. The appropriate DNA sequence must be operatively linked in the vector to an appropriate expression control sequence(s) (promoter) to direct mRNA synthesis. As representative examples of such promoters, there may be mentioned LTR or SV40 promoter, the E. coli lac or trp, the phage lambda PL promoter and other promoters known to control expression of genes in prokaryotic and eukaryotic cells or their viruses. The expression vector also includes a non-coding sequence for a ribosome binding site for translation initiation and a transcription terminator. The vector may also include appropriate sequences for amplifying expression.

The vector containing the appropriate cDNA sequence as hereinabove described, as well as an appropriate promoter or control sequence, may be employed to transform an appropriate host to permit the host to express the protein. Examples of host organisms and cells include bacterial strains (e.g., E. coli, Pseudomonas, Bacillus, Salmonella, etc.), fungi (e.g., yeasts and other fungi), animal or plant hosts (e.g., mouse, swine or animal and human tissue cells). The selection of the host is deemed to be within the scope of those skilled in the art.

It is also understood that the appropriate cDNA sequence present in the vector when introduced into a host may express part or only a portion of the protein which is encoded within the noted terminology, it being sufficient that the expressed protein be capable of eliciting an antibody or other immune response which recognizes an epitope(s) of the listed amino acid sequences.

The isolated cDNAs and/or polypeptide expressed by the host transformed by the vector may be harvested by methods which will occur to those skilled in the art and used in a vaccine for protection of a mammal, such as dogs, cattle, humans, etc., against infestations of Ixodes and non-Ixodes tick species. Such protective recombinant proteins and/or modified cDNAs are used in an amount effective to induce an immune response against such ticks and their associated pathogens and may be used in combination with a suitable physiologically acceptable carrier. The term “inducing an immune response” when used with respect to the vaccine described herein means that the vaccine prevents disease associated with a particular tick species or reduces the severity of the disease.

The carrier employed in conjunction with vaccine may be any one of a wide variety of carriers. As representative examples of suitable carriers, there may be mentioned mineral oil, synthetic polymers, etc. Carriers for vaccines are well known in the art and the selection of a suitable carrier is deemed to be within the scope of those skilled in the art. The selection of a suitable carrier is also dependent upon the manner in which the vaccine is to be administered.

The present invention provides a method of immunizing a susceptible mammal, against infestations and disease caused by ticks with the vaccine described above. For purposes of this invention, the vaccine is administered in an effective amount. The vaccine may be administered by any of the methods well known to those skilled in the art, for example, by intramuscular, subcutaneous, intraperitoneal or intravenous injection. Alternatively, the vaccine may be administered intranasally or orally. It is also to be understood that the vaccine may include active components, such as tick-borne pathogen components or adjuvants in addition to the antigen(s) or fragments hereinabove described.

The host expressing the antigen may itself be used to deliver antigen to non-human animals, by introducing killed or viable host cells that are capable of propagating in the animal. Direct incorporation of the cDNA sequences into host cells may also be used to introduce the sequences into animal cells for expression of antigen in vivo.

### BIBLIOGRAPHY

The following references are incorporated herein by reference:
Alberti E, Acosta A, Sarmiento M E, Hidalgo C, Vidal T, Fachado A, Fonte L, Izquierdo L, Infante J F, Finlay C M, Sierra G. Specific cellular and humoral immune response in Balb/c mice immunised with an expression genomic library of Trypanosoma cruzi. Vaccine 1998; 16: 608-12.
Almazán C, Kocan K M, Bergman D K, Garcia-Garcia J C, Blouin E F, de la Fuente J. Identification of protective antigens for the control of Ixodes scapularis infestations using cDNA expression library immunization. Vaccine 21, 1492-1501 (2003).
Almazán C, Kocan K M, Bergman D K, Garcia-Garcia J C, Blouin E F, de la Fuente J. Characterization of genes transcribed in an Ixodes scapularis cell line that were identified by expression library immunization and analysis of expressed sequence tags. Gene Therapy and Molecular Biology, 7:9-25 (2000).
Altschul S F, Gish W, Miller W, Myers E W, Lipman D J. Basic local alignment search tool. J Mol Biol 1990; 215: 403-10.
Barry M A, Lai W C, Johnston S A. Protection against mycoplasma infection using expression-library immunization. Nature 1995; 377: 632-5.
Black W C 4th, Piesman J. Phylogeny of hard- and soft-tick taxa (Acari: Ixodida) based on mitochondrial 16S rDNA sequences. Proc Natl Acad Sci USA 1994; 91: 10034-8.
Brayton K A, Vogel S W, Allsopp B A. Expression library immunization to identify protective antigens from Cowdria ruminantium. Ann N Y Acad Sci 1998; 849: 369-71.
Cassataro J, Velikovsky C A, Giambartolomei G H, Estein S, Bruno L, Cloeckaert A, Bowden R A, Spitz M, Fossati C A. Immunogenicity of the Brucella melitensis recombinant ribosome recycling factor-homologous protein and its cDNA. Vaccine 2002; 20: 1660-9.
de la Fuente J, Kocan K M. 2003. Advances in the identification and characterization of protective antigens for development of recombinant vaccines against tick infestations. Expert Review of Vaccines. 2:583-593.
de la Fuente J, Rodriguez M, Redondo M, Montero C, Garcia-Garcia J C, Mendez L, Serrano E, Valdes M, Enriquez A, Canales M, Ramos E, Boue O, Machado H, Lleonart R, de Armas C A, Rey S, Rodriguez J L, Artiles M, Garcia L. Field studies and cost-effectiveness analysis of vaccination with Gavac against the cattle tick Boophilus microplus. Vaccine 1998; 16: 366-73.
de la Fuente J, Rodriguez M, Montero C, Redondo M, Garcia-Garcia J C, Mendez L, Serrano E, Valdes M, Enriquez A, Canales M, Ramos E, Boue O, Machado H, Lleonart R. Vaccination against ticks (Boophilus spp.): the experience with the Bm86-based vaccine Gavac. Genet Anal 1999; 15: 143-8.
de la Fuente J, Rodriguez M, Garcia-Garcia J C. Immunological control of ticks through vaccination with Boophilus microplus gut antigens. Ann N Y Acad Sci 2000; 916: 617-21.
De Rose R, McKenna R V, Cobon G, Tennent J, Zakrzewski H, Gale K, Wood P R, Scheerlinck J P, Willadsen P. Bm86 antigen induces a protective immune response against Boophilus microplus following DNA and protein vaccination in sheep. Vet Immunol Immunopathol 1999; 71: 151-60.
de Vos S, Zeinstra L, Taoufik O, Willadsen P, Jongejan F. Evidence for the utility of the Bm86 antigen from Boophilus microplus in vaccination against other tick species. Exp Appl Acarol 2001; 25: 245-61.
Drew D R, Lightowlers M, Strugnell R A. Vaccination with plasmid DNA expressing antigen from genomic or cDNA gene forms induces equivalent humoral immune responses. Vaccine 1999; 18: 692-702.
Elad D, Segal E. Immunogenicity in calves of a crude ribosomal fraction of Trichophyton verrucosum: a field trial. Vaccine 1995; 13: 83-7.
Estrada-Peña A, Jongejan F. Ticks feeding on humans: a review of records on human-biting Ixodoidea with special reference to pathogen transmission. Exp Appl Acarol 1999; 23: 685-715.
Garcia-Garcia J C, Gonzalez I L, Gonzalez D M, Valdes M, Mendez L, Lamberti J, D'Agostino B, Citroni D, Fragoso H, Ortiz M, Rodriguez M, de la Fuente J. Sequence variations in the Boophilus microplus Bm86 locus and implications for immunoprotection in cattle vaccinated with this antigen. Exp Appl Acarol 1999; 23: 883-95.
Kofta W, Wedrychowicz H. c-DNA vaccination against parasitic infections: advantages and disadvantages. Vet Parasitol 2001; 100: 3-12.
Liyou N, Hamilton S, Elvin C, Willadsen P. Cloning and expression of ecto 5′-nucleotidase from the cattle tick Boophilus microplus. Insect Mol Biol 1999; 8: 257-66.
Liyou N, Hamilton S, Mckenna R, Elvin C, Willadsen P. Localization and functional studies on the 5′-nucleotidase of the cattle tick Boophilus microplus. Exp Appl Acarol 2000; 24: 235-46.
Manoutcharian K, Terrazas L I, Gevorkian G, Govezensky T. Protection against murine cysticercosis using cDNA expression library immunization. Immunol Lett 1998; 62: 131-6.
Melby P C, Ogden G B, Flores H A, Zhao W, Geldmacher C, Biediger N M, Ahuja S K, Uranga J, Melendez M. Identification of vaccine candidates for experimental visceral leishmaniasis by immunization with sequential fractions of a cDNA expression library. Infect Immun 2000; 68: 5595-602.
Moore R J, Lenghaus C, Sheedy S A, Doran T J. Improved vectors for expression library immunization—application to Mycoplasma hyopneumoniae infection in pigs. Vaccine 2001; 20: 115-20.
Mulenga A, Sugimoto C, Onuma M. Issues in tick vaccine development: identification and characterization of potential candidate vaccine antigens. Microbes Infect 2000; 2: 1353-61.
Munderloh U G, Wang Y L M, Chen C, Kurtti T J. Establishment, maintenance and description of cell lines from the tick Ixodes scapularis. J Parasitol 1994; 80: 533-43.
Nuttall P A. Pathogen-tick-host interactions: Borrelia burgdorferi and TBE virus. Zentralbl Bakteriol 1999; 289: 492-505.
Parola P, Raoult D. Tick-borne bacterial diseases emerging in Europe. Clin Microbiol Infect 2001; 7: 80-3.
Silva C L. The potential use of heat-shock proteins to vaccinate against mycobacterial infections. Microbes and Infection 1999; 1: 429-35.
Singh R A, Wu L, Barry M A. Generation of genome-wide CD8 T cell responses in HLA-A*0201 transgenic mice by an HIV-1 ubiquitin expression library immunization vaccine. J Immunol 2002; 168: 379-91.
Smooker P M, Setiady Y Y, Rainczuk A, Spithill T W. Expression library immunization protects mice against a challenge with virulent rodent malaria. Vaccine 2000; 18: 2533-40.
van Drunen Littel-van den Hurk S, Loehr B I, Babiuk L A. Immunization of livestock with DNA vaccines: current studies and future prospects. Vaccine 2001; 19: 2474-9.
Wikel S K, Ramachandra R N, Bergman D K, Burkot T R, Piesman J. Infestation with pathogen-free nymphs of the tick Ixodes scapularis induces host resistance to transmission of Borrelia burgdorferi by ticks. Infect Immun 1997; 65: 335-8. Willadsen P. Novel vaccines for ectoparasites. Vet Parasitol 1997; 71: 209-22.

Willadsen P, Jongejan F. Immunology of the tick-host interaction and the control of ticks and tick-borne diseases. Parasitol Today 1999; 15: 258-62.

TABLE 1
Primary screen of the I. scapularis cDNA library by ELI in mice.

| | | | | | Number of |
|---|---|---|---|---|---|
| | Number | Average ± SD | Average ± SD | Average ± SD | pools |
| | of pools | number of | number of | inhibition of | selected for |
| | screened | larvae | engorged | tick | the |
| Experimental | (Number | attached per | larvae per | infestation | secondary |
| group^{a} | of clones) | mouse^{b} | mouse^{c} | (I)^{d} | screen |
| Experiment 1 | 33 (1383) | 50 ± 13 (33-80) | 9 ± 3 (2-42) | 39 ± 55% | 6 (I > 75%) |
| | | | | (−183-87%) | |
| Vector DNA- | — | 56 ± 13 (45-67) | 16 ± 4 (5-27) | — | — |
| immunized | | | | | |
| controls for | | | | | |
| experiment 1 | | | | | |
| Experiment 2 | 33 (1322) | 56 ± 15 (29-79) | 13 ± 4 (1-27) | 27 ± 28% | 3 (I > 60%) |
| | | | | (−53-89%) | |
| Vector DNA- | — | 54 ± 18 (36-73) | 17 ± 3 (6-28) | — | — |
| immunized | | | | | |
| controls for | | | | | |
| experiment 2 | | | | | |
| ^{a}Ninety six LBA plates containing an average of 41 cDNA clones per plate were prepared. Replicas were made and clones from each plate were pooled, inoculated, grown for 2 hr in a 96 wells plate and plasmid DNA purified from each pool for ELI. Three mice per group were each immunized IM twice with 1 μg DNA/dose in 50 μl PBS two weeks apart. Two groups of 3 mice each were included as controls. One group was injected with vector DNA and the second with saline only. | | | | | |
| ^{b}Fifteen days after the last immunization, mice were infested with 100 I. scapularis larvae per mouse. Twelve hrs later, larvae that did not attach were counted to calculate the number of attached larvae per mouse and mice were transferred to new cages. | | | | | |
| ^{c}Engorged larvae dropping from each mouse were collected daily and counted after 7 days. | | | | | |
| ^{d}The inhibition of tick infestation (I) for each test group was calculated with respect to vector-immunized controls as [1 − (RLn/RLc × RLic/RLin)] × 100, where RLn is the average number of replete larvae recovered per mouse for each test group, RLc is the average number of replete larvae recovered per mouse for control group, RLic is the average number of larvae attached per mouse for control group, | | | | | |
| #and RLin is the average number of larvae attached per mouse for each test group. | | | | | |

TABLE 2
Classification of the clones in protective pools by putative protein
function according to identity to sequence databases.

| Putative protein Function | Number of clones |
|---|---|
| Biosynthetic^{a} | 2 |
| Catabolism | 4 |
| Cell adhesion | 2 |
| Cell cycle^{a} | 2 |
| Cytoskeletal^{a} | 8 |
| Defense | 2 |
| DNA structure or replication^{a} | 3 |
| Extracellular matrix | 3 |
| Endocytosis | 2 |
| Energy metabolism | 10 |
| Homeostasis | 2 |
| Morphogenetic | 9 |
| Mitochondrial^{a} | 34 |
| Protein synthesis or processing^{a,b} | 34 |
| RNA synthesis or processing^{a} | 7 |
| Heat-shock proteins | 4 |
| Signal transduction | 16 |
| Transport | 8 |
| Unknown | 199 |
| Total | 351 |

^{a}Eliminated from further screening of protective antigens. Other clones were eliminated for containing similar sequences.
^{b}Except for ribosomal proteins.

TABLE 3
Grouping of the clones according to the predicted function of
encoded proteins in sub-pools for the tertiary screen.

| Sub-pool (No. of clones) | Clone | Pool^{a} |
|---|---|---|
| Ribosomal (17) | 1A2, 1A10, 1C11 | A5 |
| | 1F6 | D1 |
| | 2B8 | A10 |
| | 2F8, 2F10 | E8 |
| | 3A10, 2C3, 3D2, 3D10 | B4 |
| | 3G9, 3G10 | E3 |
| | 4D11, 4D12, 4E7, 4F7 | F1 |
| Membrane protein (7) | 1D8, 1D11, 1E10 | D1 |
| | 2B12 | A10 |
| | 2H5 | E8 |
| | 3C9 | B4 |
| | 3G11 | E3 |
| ATPase (6) | 1A9, 1B2, 1C9 | A5 |
| | 2C9 | A10 |
| | 4A4 | C3 |
| | 4G12 | F1 |
| Cell channel/Transporter (5) | 1F4 | D1 |
| | 2H11 | E8 |
| | 4A12 | C3 |
| | 4G10, 4G11 | F1 |
| Early development-specific (4) | 1C8 | A5 |
| | 3F4 | E3 |
| | 4C7 | C3 |
| | 4G9 | F1 |
| G protein-coupled receptor (4) | 2B7, 2C12 | A10 |
| | 2F12 | E8 |
| | 4C9 | C3 |
| Growth factor receptor (3) | 2E8 | B5 |
| | 3B8, 3C8 | B4 |
| Lectin (3) | 3E10 | E3 |
| | 4B8, 4C8 | C3 |
| Vitellogenin (3) | 1F12 | D1 |
| | 4A6 | C3 |
| | 4G2 | F1 |
| Heat shock (3) | 1C10 | A5 |
| | 1F10 | D1 |
| | 3F6 | E3 |
| EGF-like (2) | 2H4 | E8 |
| | 4C10 | C3 |
| Secreted protein (2) | 2F9 | E8 |
| | 3C12 | B4 |
| Glutamine-Alanine rich (2) | 4D6, 4E6 | F1 |
| Adaptin (1) | 3E1 | E3 |
| Endopeptidase (1) | 4D8 | F1 |
| Nucleotidase (1) | 4F8 | F1 |

^{a}cDNA pools refer to positive pools after primary and secondary screens (FIG. 2A and 2B).

TABLE 4
Summary of results with I. scapularis cDNA clones.

| | | Inhibition | | |
|---|---|---|---|---|
| | | of tick | Inhibition | |
| cDNA | | infestation | of molting | Efficacy |
| clone | Predicted Protein | I (%) | M (%) | E (%) |
| 4D8 | Endopeptidase | 40*/54** | 7*/8** | 44*/58** |
| 4F8 | Nucleotidase | 50*/64** | 17*/−9** | 58*/61** |
| 1C10 | HSP70 | 17* | ND | ND |
| 4D6 | Glu-Ala-rich | 61* | 11 | 66* |
| 4E6 | Glu-Ala-rich | 20*/46** | 16** | 55** |
| 3E1 | β-adaptin (appendage | 27* | 5* | 31* |
| | region) | | | |
| 2C12 | Beta-amyloid precursor | −8*** | ND | ND |
| | protein (APP) | | | |
| 4F11 | Block of proliferation | −39*** | ND | ND |
| | Bop1 | | | |
| 3E10 | Mannose binding lectin | −48*/−10*** | ND | ND |
| 4G11 | Chloride channel | 38*** | 30 | 57 |
| 3C12 | RNA polymerase III | −104*** | ND | ND |
| 1A9, | ATPase | −57*** | ND | ND |
| 1B2, | | | | |
| 4A4 | | | | |

Mice were immunized with cDNA-containing expression plasmid DNA as described above (*) or with 100 μg/dose of recombinant protein expressed in E. coli (**). I, M and E were calculated as described above.
ND, not determined.
***Resulted in a pro-feeding activity. This effect could be due to the expression of cDNAs encoding for tick immunosuppressants, anticoagulants and other proteins with low antigenicity and a pro-feeding activity. Alternatively, they could encode for proteins homologous to host proteins with anti-tick activity, which neutralization results in a tick pro-feeding activity.

TABLE 5

RT-PCR conditions for the characterization of expression profiles
of tick protective cDNAs.

| | | | | |
|---|---|---|---|---|
| | | | Size of | Size of |
| | | PCR | cDNA | DNA |
| cDNA | RT-PCR primers (5′-3′) | conditions^{a} | band^{b} | band^{c} |

| | | | | | |
|---|---|---|---|---|---|
| 4F8 | 4F8R5: | GCGTCGTGTGGAGCATCAGCGAC | 94° C., 30 sec | 972 bp | >1,600 bp |
| | | (SEQ ID NO: 32) | 61° C., 30 sec | | |
| | 4F8-R: | TCGCAACGGACAACGGCAGGTTG | 68° C., 2 min | | |
| | | (SEQ ID NO: 33) | | | |
| 4D8 | 4D8R5: | GCTTGCGCAACATTAAAGCGAAC | 94° C., 30 sec | 577 bp | >1,600 bp |
| | | (SEQ ID NO: 34) | 56° C., 30 sec | | |
| | 4D8-R: | TGCTTGTTTGCAGATGCCCATCA | 68° C., 1 min | | |
| | | (SEQ ID NO: 35) | | | |
| 4E6 | 4E6R5: | GAAATATCTGTGAAACCAAGGCC | 94° C., 30 sec | 138 bp | ˜1,600 bp |
| | | (SEQ ID NO: 36) | 52° C., 30 sec | | |
| | 4E6U-R: | ATTGCACAACACATCATTAACTG | 68° C., 1 min | | |
| | | (SEQ ID NO: 37) | | | |

^{a}PCR conditions are indicated for denaturing, annealing and polymerization steps, respectively.
^{b}Calculated from the sequence of the cDNA insert.
^{c}Estimated from the PCR of IDE8 genomic DNA.

TABLE 6
Expression profile of mRNAs encoding for protective antigens in ticks.

| | I. scapularis | | | | | | | | | | | |
|---|---|---|---|---|---|---|---|---|---|---|---|---|
| RNA | | | | | | salivary | I. | | | D. | R. | B. |
| source | IDE8 | eggs | larvae | nymphs | guts | glands | pacificus | I. ricinus | A. americanum | variabilis | sanguineus | microplus |
| Clone | mRNA expression | | | | | | | | | | | |
| 4F8 | + | + | + | + | + | + | − | + | − | − | − | − |
| 4D8 | + | + | + | + | + | + | + | + | + | + | + | + |
| 4E6 | + | + | + | + | + | + | + | + | + | − | − | − |

Expression of mRNAs encoding for tick protective antigens 4F8, 4D8 and 4E6 was analyzed by RT-PCR in different tick species using the primers derived from I. scapularis sequences.

TABLE 7
Results of vaccination with recombinant tick protective antigens
on I. scapularis, D. variabilis and A. americanum nymphs.

| | Inhibition of tick | | | Reduction in the weight of | | | | |
|---|---|---|---|---|---|---|---|---|
| | nymphal infestation (%)^{a} | | | engorged nymphs (%)^{b} | | | | |
| Tick species | 4D8 | 4F8 | 4E6 | All | 4D8 | 4F8 | 4E6 | All |
| I. scapularis | 35 | 39 | 0 | 63 | 0 | 0 | 0 | 0 |
| D. variabilis | 22 | 0 | 5 | 8 | 32 | 0 | 27 | 0 |
| A. americanum | 17 | 9 | 36 | 12 | 3 | 1 | 0 | 16 |

^{a}Determined with respect to the number of engorged nymphs recovered from the control group as described above for tick larval infestations.
^{b}Average weight per engorged nymph with respect to the weight of nymphs collected from the control group.

In view of the above, it will be seen that the several objectives of the invention are achieved and other advantageous results attained. As various changes could be made in the above DNA molecules, proteins, etc. without departing from the scope of the invention, it is intended that all matter contained in the above description or shown in the accompanying drawings shall be interpreted as illustrative and not in a limiting sense. While the invention has been described with a certain degree of particularity, it is understood that the invention is not limited to the embodiment(s) set for herein for purposes of exemplification, but is to be limited only by the scope of the attached claim or claims, including the full range of equivalency to which each element thereof is entitled.

1. An isolated polypeptide selected from the group consisting of tick derived polypeptides designated 4D8, 4F8 and 4E6.

2. A multi species anti-tick vaccine composition comprising the polypeptide of claim 1 and a pharmaceutically acceptable carrier or diluent.

3. The isolated polypeptide of claim 1, wherein the polypeptide comprises the tick derived polypeptide 4D8.

4. An isolated polypeptide comprising the amino acid sequence of at least one of SEQ ID NO:4, SEQ ID NO:27, SEQ ID NO:29, SEQ ID NO: 31, or SEQ ID NO:39.

5. A multi species anti-tick vaccine composition comprising the polypeptide of claim 4 and a pharmaceutically acceptable carrier or diluent.

6. The polypeptide of claim 4, wherein the polypeptide comprises the amino acid sequence of SEQ ID NO:4.

7. The polypeptide of claim 4, wherein the polypeptide comprises the amino acid sequence of SEQ ID NO:27.

8. The polypeptide of claim 4, wherein the polypeptide comprises the amino acid sequence of SEQ ID NO:29.

9. The polypeptide of claim 4, wherein the polypeptide comprises the amino acid sequence of SEQ ID NO:31.

10. The polypeptide of claim 4, wherein the polypeptide comprises the amino acid sequence of SEQ ID NO:39.

11. An isolated polypeptide comprising an amino acid sequence that is at least 95% homologous to at least one of SEQ ID NO:4, SEQ ID NO:27, SEQ ID NO:29, SEQ ID NO: 31, or SEQ ID NO:39.

12. An isolated cDNA molecule which encodes a tick derived antigenic polypeptide, said molecule having a nucleotide sequence that is at least 95% homologous to at least residues 1-117 of SEQ ID NO: 1.

13. An expression vector comprising the isolated cDNA molecule of claim 12.

14. An isolated cell transformed by the expression vector of claim 13.

15. A multi species anti-tick vaccine comprising an effective immunizing amount of an immunogen selected from the group consisting of (i) the isolated cDNA molecule of claim 12, (ii) the polypeptide encoded by the isolated cDNA molecule of claim 12, and (iii) a combination of the isolated cDNA molecule of claim 12 and the encoded polypeptide, and further comprising a pharmaceutically acceptable carrier or diluent.

16. A method of inducing an immune response in a mammal against multi species tick infestation and their associated pathogens, comprising administering to the mammal the vaccine of claim 15.

17. The polypeptide encoded by the isolated cDNA molecule of claim 12.

18. An isolated cDNA molecule which encodes a tick derived antigenic polypeptide, said molecule having a nucleotide sequence that is at least 95% homologous to at least residues 80-575 of SEQ ID NO: 3.

19. An expression vector comprising the isolated cDNA molecule of claim 18.

20. An isolated cell transformed by the expression vector of claim 19.

21. A multi species anti-tick vaccine comprising an effective immunizing amount of an immunogen selected from the group consisting of (i) the isolated cDNA molecule of claim 18, (ii) the polypeptide encoded by the isolated cDNA molecule of claim 18, and (iii) a combination of the isolated cDNA molecule of claim 18 and the encoded polypeptide, and further comprising a pharmaceutically acceptable carrier or diluent.

22. A method of inducing an immune response in a mammal against multi species tick infestation and their associated pathogens, comprising administering to the mammal the vaccine of claim 21.

23. The polypeptide encoded by the isolated cDNA molecule of claim 18.

24. An isolated cDNA molecule which encodes a tick derived antigenic polypeptide, said molecule having a nucleotide sequence that is at least 95% homologous to at least residues 10-951 of SEQ ID NO: 5.

25. An expression vector comprising the isolated cDNA molecule of claim 24.

26. An isolated cell transformed by the expression vector of claim 25.

27. A multi species anti-tick vaccine comprising an effective immunizing amount of an immunogen selected from the group consisting of (i) the isolated cDNA molecule of claim 24, (ii) the polypeptide encoded by the isolated cDNA molecule of claim 24, and (iii) a combination of the isolated cDNA molecule of claim 24 and the encoded polypeptide, and further comprising a pharmaceutically acceptable carrier or diluent.

28. A method of inducing an immune response in a mammal against multi species tick infestation and their associated pathogens, comprising administering to the mammal the vaccine of claim 27.

29. The polypeptide encoded by the isolated cDNA molecule of claim 24.

30. An isolated cDNA molecule which encodes a tick derived antigenic polypeptide, said molecule having a nucleotide sequence that is at least 95% homologous to SEQ ID NO: 26.

31. An expression vector comprising the isolated cDNA molecule of claim 30.

32. An isolated cell transformed by the expression vector of claim 31.

33. A multi species anti-tick vaccine comprising an effective immunizing amount of an immunogen selected from the group consisting of (i) the isolated cDNA molecule of claim 30, (ii) the polypeptide encoded by the isolated cDNA molecule of claim 30, and (iii) a combination of the isolated cDNA molecule of claim 30 and the encoded polypeptide, and further comprising a pharmaceutically acceptable carrier or diluent.

34. A method of inducing an immune response in a mammal against multi species tick infestation and their associated pathogens, comprising administering to the mammal the vaccine of claim 33.

35. The polypeptide encoded by the isolated cDNA molecule of claim 30.

36. An isolated cDNA molecule which encodes a tick derived antigenic polypeptide, said molecule having a nucleotide sequence that is at least 95% homologous to SEQ ID NO: 28.

37. An expression vector comprising the isolated cDNA molecule of claim 36.

38. An isolated cell transformed by the expression vector of claim 37.

39. A multi species anti-tick vaccine comprising an effective immunizing amount of an immunogen selected from the group consisting of (i) the isolated cDNA molecule of claim 36, (ii) the polypeptide encoded by the isolated cDNA molecule of claim 36, and (iii) a combination of the isolated cDNA molecule of claim 36 and the encoded polypeptide, and further comprising a pharmaceutically acceptable carrier or diluent.

40. A method of inducing an immune response in a mammal against multi species tick infestation and their associated pathogens, comprising administering to the mammal the vaccine of claim 39.

41. The polypeptide encoded by the isolated cDNA molecule of claim 36.

42. An isolated cDNA molecule which encodes a tick derived antigenic polypeptide, said molecule having a nucleotide sequence that is at least 95% homologous to SEQ ID NO: 30.

43. An expression vector comprising the isolated cDNA molecule of claim 42.

44. An isolated cell transformed by the expression vector of claim 43.

45. A multi species anti-tick vaccine comprising an effective immunizing amount of an immunogen selected from the group consisting of (i) the isolated cDNA molecule of claim 42, (ii) the polypeptide encoded by the isolated cDNA molecule of claim 42, and (iii) a combination of the isolated cDNA molecule of claim 42 and the encoded polypeptide, and further comprising a pharmaceutically acceptable carrier or diluent.

46. A method of inducing an immune response in a mammal against multi species tick infestation and their associated pathogens, comprising administering to the mammal the vaccine of claim 45.

47. The polypeptide encoded by the isolated cDNA molecule of claim 42.

48. An isolated cDNA molecule which encodes a tick derived antigenic polypeptide, said molecule having a nucleotide sequence that is at least 95% homologous to SEQ ID NO: 38.

49. An expression vector comprising the isolated cDNA molecule of claim 48.

50. An isolated cell transformed by the expression vector of claim 49.

51. A multi species anti-tick vaccine comprising an effective immunizing amount of an immunogen selected from the group consisting of (i) the isolated cDNA molecule of claim 48, (ii) the polypeptide encoded by the isolated cDNA molecule of claim 48, and (iii) a combination of the isolated cDNA molecule of claim 48 and the encoded polypeptide, and further comprising a pharmaceutically acceptable carrier or diluent.

52. A method of inducing an immune response in a mammal against multi species tick infestation and their associated pathogens, comprising administering to the mammal the vaccine of claim 51.

53. The polypeptide encoded by the isolated cDNA molecule of claim 48.
